SEQUENCE LISTING (24 sequences)

SEQ ID NO: 1 - DNA, 14 bp, Artificial
  Synthetic oligonucleotide
cccctttctttttt

SEQ ID NO: 2 - DNA, 14 bp, Artificial
  Synthetic oligonucleotide
cccctttctttttt

SEQ ID NO: 3 - DNA, 14 bp, Artificial
  Synthetic oligonucleotide
cccctttctttttt

SEQ ID NO: 4 - DNA, 14 bp, Artificial
  Synthetic oligonucleotide
cccctttctttttt

SEQ ID NO: 5 - DNA, 14 bp, Artificial
  Synthetic oligonucleotide
cccctttctttttt

SEQ ID NO: 6 - DNA, 14 bp, Artificial
  Synthetic oligonucleotide
cccctttctttttt

SEQ ID NO: 7 - DNA, 14 bp, Artificial
  Synthetic oligonucleotide
cccctttctttttt

SEQ ID NO: 8 - DNA, 14 bp, Artificial
  Synthetic oligonucleotide
cccctttctttttt

SEQ ID NO: 9 - DNA, 14 bp, Artificial
  Synthetic oligonucleotide
cccctttctttttt

SEQ ID NO: 10 - DNA, 14 bp, Artificial
  Synthetic oligonucleotide
cccctttctttttt

SEQ ID NO: 11 - DNA, 14 bp, Artificial
  Synthetic oligonucleotide
cccctttctttttt

SEQ ID NO: 12 - DNA, 14 bp, Artificial
  Synthetic oligonucleotide
cccctttctttttt

SEQ ID NO: 13 - DNA, 14 bp, Artificial
  Synthetic oligonucleotide
cccctttctttttt

SEQ ID NO: 14 - DNA, 14 bp, Artificial
  Synthetic oligonucleotide
cccctttctttttt

SEQ ID NO: 15 - DNA, 14 bp, Artificial
  Synthetic oligonucleotide
cccctttctttttt

SEQ ID NO: 16 - DNA, 14 bp, Artificial
  Synthetic oligonucleotide
cccctttctttttt

SEQ ID NO: 17 - DNA, 14 bp, Artificial
  Synthetic oligonucleotide
cccctttctttttt

SEQ ID NO: 18 - DNA, 14 bp, Artificial
  Synthetic oligonucleotide
cccctttctttttt

SEQ ID NO: 19 - DNA, 14 bp, Artificial
  Synthetic oligonucleotide
ggggaaagaaaaaa

SEQ ID NO: 20 - RNA, 14 nt, Artificial
  Synthetic oligonucleotide
ggggaaagaaaaaa

SEQ ID NO: 21 - DNA, 17 bp, Artificial
  Synthetic DNA
ctgcccctttctttttt

SEQ ID NO: 22 - DNA, 17 bp, Artificial
  Synthetic oligonucleotide
ctgccccttactttttt

SEQ ID NO: 23 - DNA, 17 bp, Artificial
  Synthetic oligonucleotide
ctgccccttcctttttt

SEQ ID NO: 24 - DNA, 17 bp, Artificial
  Synthetic oligonucleotide
ctgccccttgctttttt

(19) United States
(12) Patent Application Publication
(10) Pub. No.: US 2011/0130557 A1
(43) Pub. Date: Jun. 2, 2011

(54) INTERCALATING TRIPLEXES AND DUPLEXES USING ARYL NAPHTHOIMIDAZOL AND PROCESS FOR THE PREPARATION THEREOF

(76) Inventors: Erik Bjerregaard Pedersen, Odense (DK); Amany Mostafa Ahmed Osman, Shebin El-Kom (EG); Per Trolle Jorgensen, Odense (DK); Niels Bomholt, Otterup (DK)

(21) Appl. No.: 12/991,058

(22) PCT Filed: May 6, 2009
(86) PCT No.: PCT/EP09/55507

§ 371 Date: Dec. 15, 2010

Related U.S. Application Data
(60) Provisional application No. 61/078,063, filed on Jul. 3, 2008.

(30) Foreign Application Priority Data
May 7, 2008 (DK) PA 2008 00648

(51) Int. Cl. C07H 21/04

(52) U.S. Cl. 536/24.5

(57) ABSTRACT

There is provided an intercalating oligonucleotide for stabilizing natural or modified DNA and RNA triplexes, duplexes and hybrids thereof having the general structure (I) triplex forming oligonucleotides of the invention are capable of binding specifically to double stranded target nucleic acids and are therefore of interest for modulation of the activity of target nucleic acids and also detection of target nucleic acids.

### BACKGROUND OF THE INVENTION

The ability of triplex-forming oligonucleotides (TFOs) to interact specifically with polypurine/polypyrimidine double-stranded DNA forming triplexes has shown them as candidates for regulation of transcription of genomic DNA in the so-called antigene strategy.^{[1-7]} Moreover, TFOs induce gene recombination and repairing genetic defects in mammalian cells.^{[8-10]} However, in many cases triplexes are thermodynamically less stable than corresponding duplexes. For this reason an enormous number of oligodeoxynucleotides (ODN) have been developed, either by modifying the nucleobase,^{[11-13]} the sugar part,^{14-19]} or the phosphate backbone^{[20-27]} to improve triplex stabilization. The triplex stabilization can also be achieved by insertion of different intercalating agents. Recently, the extraordinary stable Hoogsteen type triplexes and duplexes have been observed, when the intercalator (R)-1-O-[4-(1-pyrenylethynyl)benzyl]-glycerol (W, TINA, FIG. 1) was inserted as a bulge in the middle of a TFO.²⁸ Meanwhile, there is a need to provide further stable intercalators.

WO06125447A2^{[40]} discloses intercalator oligonucleotides capable of being incorporated into the backbone of an oligonucleotide or an oligonucleotide analogue. The oligonucleotides have a linker (L) bonded to an aromatic or heteroaromatic ring (Ar) that via a single bond is attached to W (2-6 condensed aromatic or heteroaromatic rings). The oligonucleotides show increased stability (higher Tm) under hybridization with especially double stranded DNA. Specifically, two oligonucleotides are disclosed, wherein methylene (linker) is bonded to the backbone, Ar is triazole that is attached to a condensed ring system (pyrene and naphthalimid) via a single bond.

TIMOFEEV et al^{[41]} discloses intercalator oligonucleotides, wherein compound 4 is incorporated in a nucleic acid sequence. The presence of the increased stability (higher Tm) under hybridization with especially double stranded DNA. The intercalator pseudonucleotides are thus capable of being incorporated into the backbone of an oligonucleotide or an oligonucleotide analogue and increase the stability thereof by increasing the Tm with 8.1° C. The compound 4 is incorporated in a nucleic acid sequence so that a linker being bonded to the two oligomeric fragments is also bonded to a benzene ring that is further bonded via a single bond to a condensed ring system.

The present invention aims at providing alternative intercalator structures to those of the prior art.

### SUMMARY OF THE INVENTION

The present inventors have surprisingly found that inserting 2-phenyl or 2-naphth-1-yl-phenanthroimidazole intercalators (X and Y, respectively, FIG. 1) as bulges into triplex-forming oligonucleotides, both intercalators show extraordinary high thermal stability of the corresponding Hoogsteen-type triplexes and Hoogsteen-type parallel duplexes with high discrimination to Hoogsteen mismatches. Molecular modeling shows that the phenyl or the naphthyl ring stacks with the nucleobases in the TFO, while the phenanthroimidazol moiety stacks with the base pairs of the dsDNA. DNA-strands containing the intercalator X show higher thermal triplex stability than DNA-strands containing the intercalator Y. The difference can be explained by a lower degree of planarity of the intercalator in the case of naphthyl. It was also observed that triplex stability was considerably reduced when the intercalators X or Y was replaced by 2-(naphthlen-1-yl)imidazole. This confirms intercalation as the important factor for triplex stabilization and it rules out an alternative complexation of protonated imidazole with two phosphate groups. The intercalating nucleic acid monomers X and Y were obtained via a condensation reaction of 9,10-phenanthrenequinone (4) with (S)-4-(2-(2,2-dimethyl-1,3-dioxolan-4-yl)ethoxy)benzaldehyde (3a) or (S)-4-(2-(2,2-dimethyl-1,3-dioxolan-4-yl)ethoxy)-1-naphthaldehyde (3b), respectively, in the presence of acetic acid and ammonium acetate. The required monomers for DNA synthesis using amidite chemistry were obtained by standard deprotection of the hydroxy groups followed by 4,4′-dimethoxytritylation and phosphitylation.

Accordingly, the present invention provides an intercalating oligonucleotide for stabilizing natural or modified DNA and RNA triplexes, duplexes and hybrids thereof having the general structure (I):

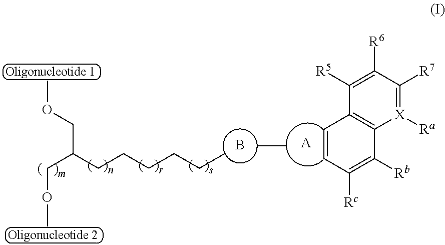

wherein

R^{a} and R^{b} together form

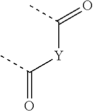

R^{c} is H

or

R^{b} and R^{c} together form

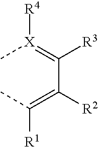

R^{a}═R⁸

wherein A is a 5-, 6-, or 7-membered heteroaromatic ring, containing at least one heteroatom selected from nitrogen, oxygen and sulfur, especially one nitrogen atom and at least one further heteroatom selected from nitrogen, substituted nitrogen, oxygen and sulfur,

wherein B is a monocyclic or polycyclic aromatic ring systems optionally selected from the group of

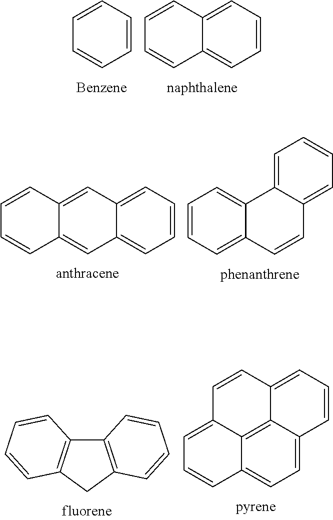

and monocyclic or bicyclic heteromatic ring systems optionally selected from the group of 5-membered aromatic heterocyclic rings and

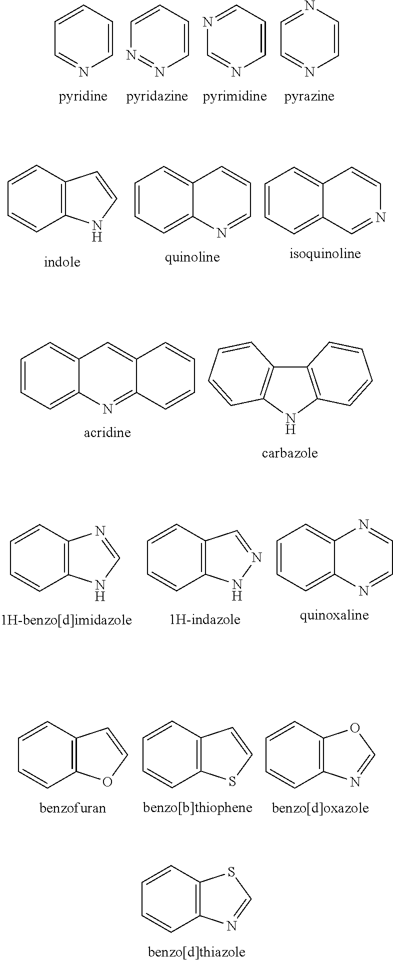

wherein

P and R are independently of each other selected from the group consisting of O, S, NR⁹, —CH₂, —CH—, —C≡C—, wherein R⁹ is hydrogen, methyl, ethyl, or hydroxyl,

m is 0 or 1, n, r, s are independently of each other 0, 1, 2 or 3, especially 0, 1 or 2,

Oligonucleotide 1 and Oligonucleotide 2 are defined independently of each other oligonucleotide consisting of subunits of DNA, RNA, PNA, HNA, MNA, ANA, FANA, LNA, CAN, INA, CeNA, TNA, (2′-NH)-TNA, (3′-NH)-TNA, α-L-Ribo-LNA, α-L-Xylo-LNA, β-D-Ribo-LNA, β-D-Xylo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-RRNA, 2′-OR-RNA, 2′-AE-RNA, α-L-RNA, β-D-RNA, and modifications thereof,

R¹, R², R³, R⁴ R⁵, R⁶, R⁷ and R⁸ are independently of each other hydrogen, halogen, C₁-C₁₈alkyl, C₁-C₁₈alkyl which is substituted by E and/or interrupted by D, C₂-C₁₈alkenyl, C₂-C₁₈alkynyl, C₁-C₁₈alkoxy, C₁-C₁₈alkoxy which is substituted by E and/or interrupted by D, C₆-C₂₄aryl, C₆-C₂₄aryl which is substituted by G, C₂-C₂₀heteroaryl, C₂-C₂₀heteroaryl which is substituted by G, C₇-C₂₅arakyl,

or two substituents R¹ and R², R² and R³, R³ and R⁴, R⁵ and R⁶, R⁶ and R⁷, R⁷ and R⁸ which are adjacent to each other, together form a group

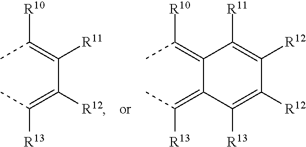

or two substituents R⁴ and R⁸, which are adjacent to each other, together form a group

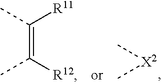

wherein R¹⁰, R¹¹, R¹², R¹³ are independently of each other hydrogen, halogen, C₁-C₁₈alkyl, C₁-C₁₈alkyl which is substituted by E and/or interrupted by D, C₂-C₁₈alkenyl; C₂-C₁₈alkynyl, C₁-C₁₈alkoxy, C₁-C₁₈alkoxy which is substituted by E and/or interrupted by D, C₆-C₂₄aryl, C₆-C₂₄aryl which is substituted by G, C₂-C₂₀heteroaryl, C₂-C₂₀heteroaryl which is substituted by G, C₇-C₂₅aralkyl;

X² is O, S, C(R¹⁴)(R¹⁵), or N—R¹⁶, wherein R¹⁶ is hydrogen, hydroxyl, C₁-C₁₈alkyl, C₁-C₁₈alkyl which is substituted by E and/or interrupted by D, C₂-C₁₈alkenyl, C₂-C₁₈alkynyl which is substituted by E and/or interrupted by D, C₁-C₁₈alkoxy, C₁-C₁₈alkoxy which is substituted by E and/or interrupted by D, C₁-C₁₈aminoalkyl, C₁-C₁₈aminoalkyl which is substituted by E and/or interrupted by D, C₅-C₁₈cycloalkyl, C₅-C₁₈cycloalkyl which is substituted by E and/or interrupted by D, C₆-C₁₈aryl, C₂-C₂₀heteroaryl, C₆-C₁₈aryl, or C₂-C₂₀heteroaryl, which are substituted by C₁-C₁₈alkyl, or C₁-C₁₈alkoxy; C₁-C₁₈alkyl; or C₁-C₁₈alkyl which is interrupted by —O—,

R¹⁴ and R¹⁵ together form a group of formula ═CR¹⁷R¹⁸, wherein R¹⁷ and R¹⁸ are independently of each other hydrogen, C₁-C₁₈alkyl, C₁-C₁₈alkyl which is substituted by E and/or interrupted by D, C₆-C₂₄aryl, C₆-C₂₄aryl which is substituted by G, C₂-C₂₀heteroaryl, or C₂-C₂₀heteroaryl which is substituted by G, or R¹⁴ and R¹⁵ together form a five or six membered ring, which can be substituted by C₁-C₁₈alkyl, C₁-C₁₈alkyl which is substituted by E and/or interrupted by D, C₆-C₂₄aryl, C₆-C₂₄aryl which is substituted by G, C₂-C₂₀heteroaryl, or C₂-C₂₀heteroaryl which is substituted by G, C₂-C₁₈alkenyl; C₂-C₁₈alkynyl, C₁-C₁₈alkoxy, C₁-C₁₈alkoxy which is substituted by E and/or interrupted by D, C₇-C₂₅aralkyl, or —C(═O)—R¹⁹, wherein R¹⁹ is hydrogen, C₆-C₁₈aryl, C₆-C₁₈aryl which is substituted by C₁-C₁₈alkyl, or C₁-C₁₈alkoxy, C₁-C₁₈alkyl, or C₁-C₁₈alkyl which is interrupted by —O—,

D is —CO—, —S—, —SO—, —SO₂, —O—, —NR²⁰—, —SiR²¹R²²—, —POR²³—, —CR²⁴═CR²⁵—, or —C≡C—; and

E is —OR²⁶, —SR²⁶, —COR²⁶, —NR²⁰R²⁷, CN, or halogen,

G is E, C₁-C₁₈alkyl, C₁-C₁₈alkyl which is interrupted by D, C₁-C₁₈alkoxy, or C₁-C₁₈alkoxy which is substituted by E and/or interrupted by D, wherein

R²⁰, R²⁴, R²⁵, R²⁷ are independently of each other hydrogen, C₁-C₁₈alkyl, C₆-C₁₈aryl, C₆-C₁₈aryl which is substituted by C₁-C₁₈alkyl, or C₁-C₁₈alkoxy, C₁-C₁₈alkyl, or C₁-C₁₈alkyl which is interrupted by —O—, or

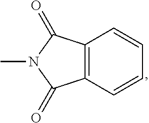

R²⁰ and R²⁷ together form a five or six membered ring, in particular

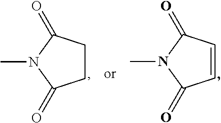

R²¹, R²² and R²³ are independently of each other C₁-C₁₈alkyl, C₆-C₁₈aryl, or C₆-C₁₈aryl, which is substituted by C₁-C₁₈alkyl, and

R²⁶ is independently of each other hydrogen, C₁-C₁₈alkyl, C₆-C₁₈aryl, C₆-C₁₈aryl which is substituted by C₁-C₁₈alkyl, or C₁-C₁₈alkoxy, C₁-C₁₈alkyl, or C₁-C₁₈alkyl which interrupted by —O—,

X is C or N with the proviso that when X is CH or N then the nitrogen atom is unsubstituted, and

Y is O or N—R²⁸, wherein R²⁸ is hydrogen, methyl, ethyl, hydroxyl, alkyl, substituted alkyl, alkoxy, substituted alkoxy, aminoalkyl, substituted aminoalkyl, cycloalkyl, substituted cycloalkyl, alkenyl, substituted alkenyl, alkynyl, substituted alkynyl, aryl, substituted aryl, heterocyclic, and substituted heterocyclic.

When reference is made to hetero, such as hetero-aryl, it means N, O, and S.

In a preferred embodiment the present invention provides intercalating oligonucleotides having having any one of the general structures (IIa-IId):

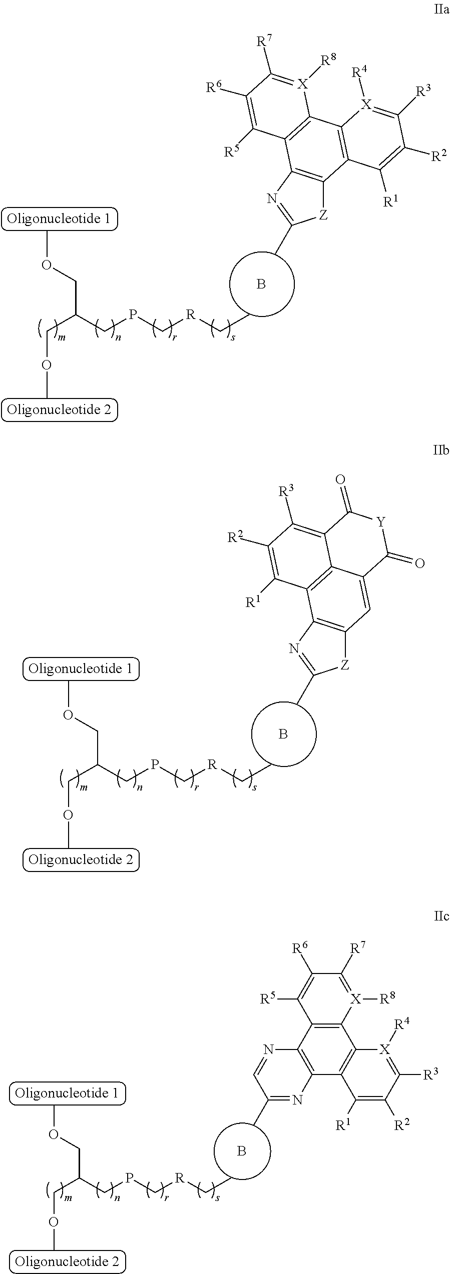

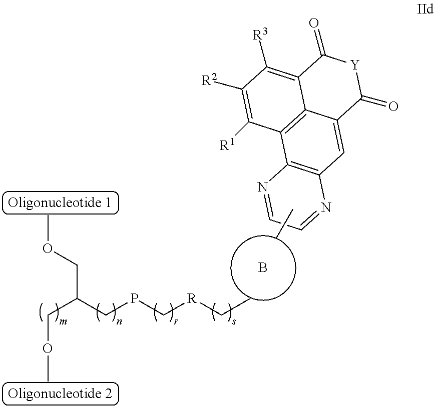

In still another embodiment there is provided an intercalating oligonucleotide having the structures (Va-Vh):

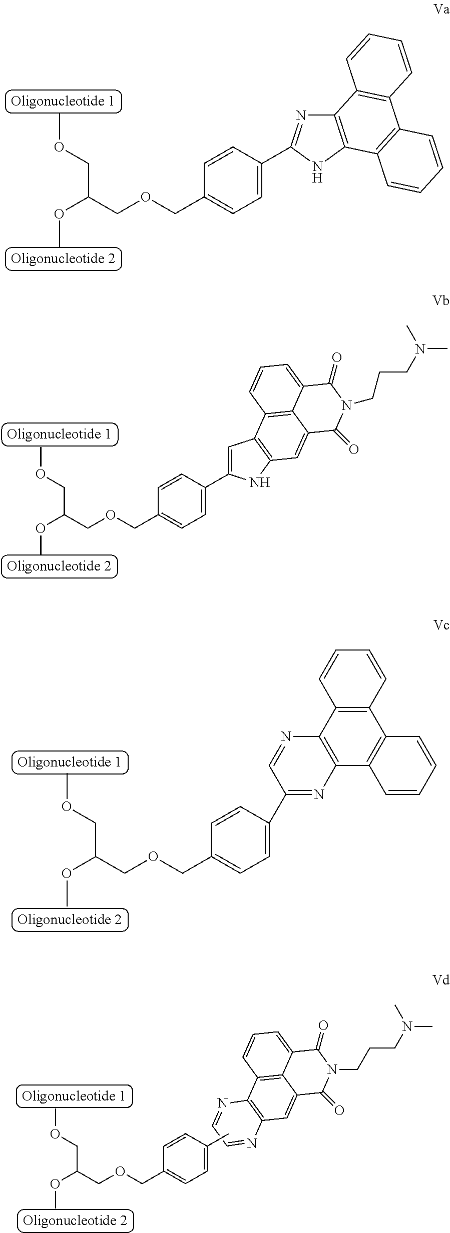

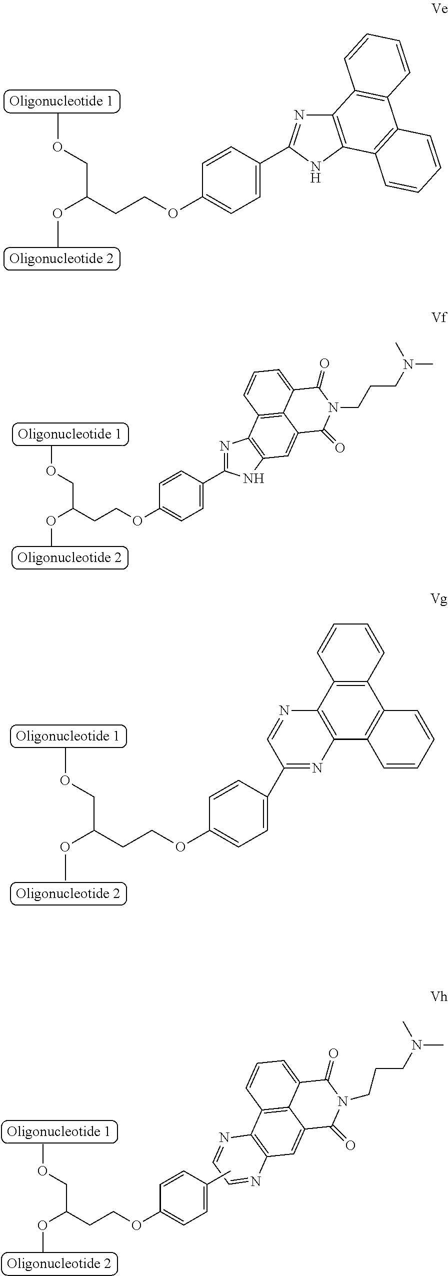

The present invention further provides a pharmaceutical composition suitable for use in antisense therapy and antigene therapy, said composition comprising an intercalating oligonucleotide of the present invention.

When inserting 2-phenyl or 2-naphth-1-yl-phenanthroimidazole intercalators (X and Y, respectively) as bulges into triplex-forming oligonucleotides, both intercalators show extraordinary high thermal stability of the corresponding Hoogsteen-type triplexes and Hoogsteen-type parallel duplexes with high discrimination to Hoogsteen mismatches. Molecular modeling shows that the phenyl or the naphthyl ring stacks with the nucleobases in the TFO, while the phenanthroimidazol moiety stacks with the base pairs of the dsDNA. DNA-strands containing the intercalator X show higher thermal triplex stability than DNA-strands containing the intercalator Y. The difference can be explained by a lower degree of planarity of the intercalator in the case of naphthyl. It was also observed that triplex stability was considerably reduced when the intercalators X or Y was replaced by 2-(naphthlen-1-yl)imidazole. This confirms intercalation as the important factor for triplex stabilization and it rules out an alternative complexation of protonated imidazole with two phosphate groups. The intercalating nucleic acid monomers X and Y were obtained via a condensation reaction of 9,10-phenanthrenequinone (4) with (S)-4-(2-(2,2-dimethyl-1,3-dioxolan-4-yl)ethoxy)benzaldehyde (3a) or (S)-4-(2-(2,2-dimethyl-1,3-dioxolan-4-yl)ethoxy)-1-naphthaldehyde (3b), respectively, in the presence of acetic acid and ammonium acetate. The required monomers for DNA synthesis using amidite chemistry were obtained by standard deprotection of the hydroxy groups followed by 4,4′-dimethoxytritylation and phosphitylation.

### BRIEF DESCRIPTION OF THE DRAWINGS

FIG. 1 shows the synthesized intercalators X, Y, Z and V with the reference intercalator W (TINA).

FIG. 2 shows first derivatives plots of triplex melting (up and down) for ON3 and ON2 incorporating monomer X and W respectively, recorded at 260 nm versus increasing temperature (1° C./min) in 20 mM sodium cacodylate, 100 mM NaCl, 10 mM MgCl₂, pH 6.0.

FIG. 3 shows fluorescence emission spectra of ON3 incorporating monomer X upon excitation at 373 nm and pH 6.0. A) ON3 forming parallel triplex and mismatched triplexes. B) ON3 forming parallel duplex and antiparallel duplex.

FIG. 4 shows representative low-energy structures of intercalator X (left) and Y (right).

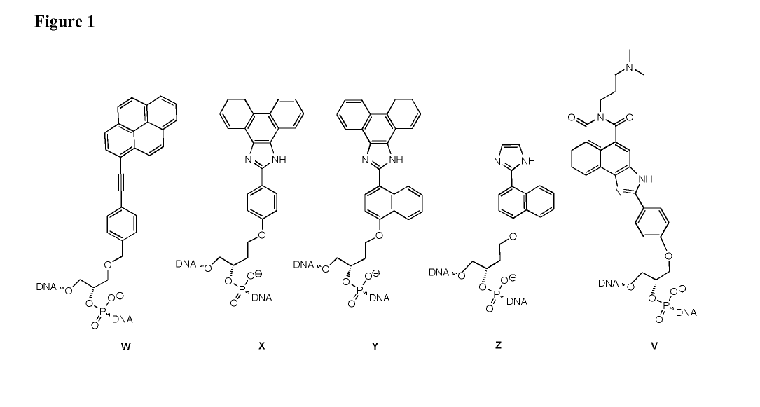

### DETAILED DESCRIPTION OF THE INVENTION

The synthetic route towards the intercalating nucleic acid monomers (6a,b) is shown in (Scheme 1). The key intermediates 3a,b were synthesized from (5)-2-(2,2-dimethyl-1,3-dioxolan-4-yl)ethanol (1) by reaction with 4-hydroxybenzaldehyde (2a) or 4-hydroxy- 1-naphthaldehyde (2b) under Mitsunobu conditions^{[32]} (DEAD, THF, Ph₃P) in high yields 81% and 92%, respectively (Scheme 1). Subsequent treatment of 3a,b with phenanthrene-9,10-dione (4) and ammonium acetate in hot glacial acetic acid according to the procedure of Krebs and Spanggaard^{[33]} afforded the monomers 6a,b. When starting from 3a the product mixture was separated by silica gel column chromatography to afford the deprotected (S)-4-(4-(1H-phenanthro[9,10-d]imidazol-2-yl)phenoxy)butane-1,2-diol (6a) in 72% yield and a minor amount of the corresponding diol (5) still protected with an isopropylidene group. Due to exchange of the imidazole protons, a line broadening was observed in the ¹H-NMR spectrum of (5). This resulted in a broad singlet for the neighboring protons in the phenanthrene ring at C-4 and C-11. The corresponding compound (S)-4-(4-(1H-phenanthro [9,10-d]imidazol-2-yl)naphalen-1-yloxy)butane-1,2-diol (6b) was isolated fully deprotected by precipitation in 80% yield without chromatographic purification. The primary hydroxy group of compounds (6a,b) was protected by reaction with 4,4′-dimethoxytrityl chloride (DMT-Cl) in anhydrous pyridine at room temperature under a N₂ atmosphere. Silica gel purification afforded the DMT-protected compounds 7a,b in 79% and 56% yield, respectively. The secondary hydroxy group of these compounds was phosphitylated overnight with 2-cyanoethyl N,N,N′,N′-tetraisopropyl phosphorodiamidite in the presence of diisopropyl ammonium tetrazolide as activator in anhydrous CH₂Cl₂ to afford 8a,b in 86% and 81% yield, respectively (Scheme 1).

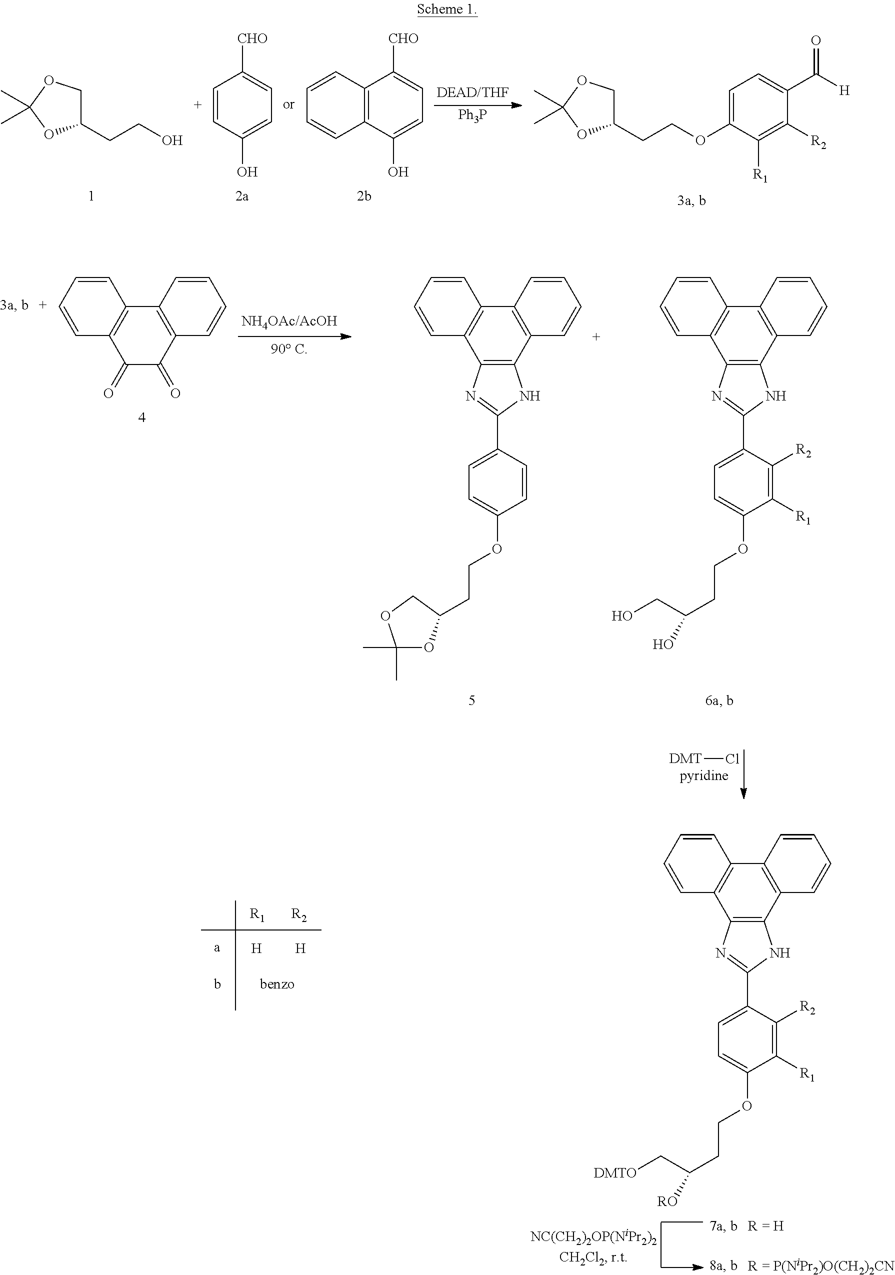

It was believed that the corresponding imidazolyl amidite derivative 13 without the phenanthrene ring system could be easily obtained from the corresponding monomer 10 (Scheme 2). In order to synthesize the monomer 10, compound 3b was deprotected with 80% aqueous acetic acid to give (S)-4-(3,4-dihydroxybutoxy)-1-naphthaldehyde (9) in 100% yield. This compound was reacted in ethanol and MeCN at 0° C. with a solution of 40% glyoxal in water and 20 M ammonium hydroxide overnight to afford (S)-4-(4-(1H-imidazol-2-yl)naphthalene-1-yloxy)butan-1,2-diol (10) in 44% yield in analogy with the procedure of Nakumura et al.^{[34]} Unfortunately, the subsequent attempt to make the DMT protected compound 12 failed although a variety of procedures were investigated. Therefore, it was decided to change the synthetic strategy. Instead, the primary hydroxyl group of compound 9 was DMT-protected to afford the compound 11 in 60% yield after purification by column chromatography. The imadazolyl derivative 12 was then obtained in 32% yield from compound 11 using the same reaction conditions as used for converting compound 9 into compound 10. Finally, the amidite 13 was obtained in 81% yield by a standard phosphitylation reaction of compound 12.

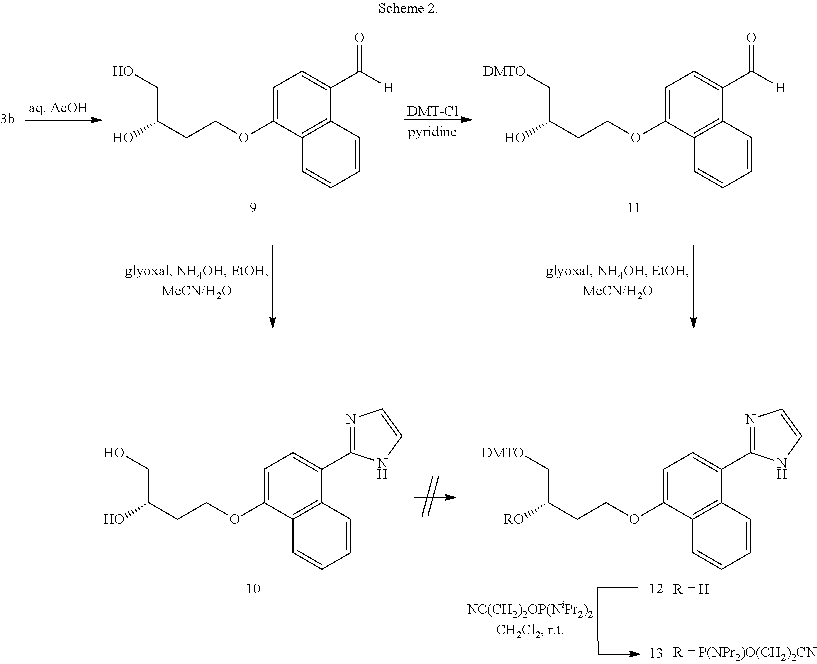

The obtained phosphoramidites 8a,b and 13 were incorporated into a 14-mer oligonucleotides by a standard phosphoramidite protocol on an automated DNA synthesizer. However, an extended coupling time (10 min), in the oligonucleotide synthesis as was used for the amidite of the natural nucleosides. All modified ODNs were purified by reversed-phase HPLC, and confirmed by MALDI-TOF-MS analysis. The purity of the final sequences was determined by ion-exchange HPLC (IE-HPLC) to be more than 90%.

The thermal stabilities of parallel triplexes and duplexes as well as antiparallel DNA/DNA and DNA/RNA duplexes containing the intercalators X, Y and Z were evaluated by thermal denaturation experiments. The thermal melting studies of X and Y were compared with the previously published data for the intercalator W (TINA)^{[28a]} as shown in Tables 1, 2, and 3. The melting temperatures (Tₘ, ° C.) were determined as the first derivatives of melting curves. Since protonated cytosine only is able to form Hoogsteen bonds, thermal stability of parallel triplexes using the synthesized oligonucleotides towards the duplex (D1)^{[35]} was assessed both at pH 6.0 and pH 7.2, the ultimate goal being to find triplex formation at physiological pH conditions. Thermal stability of the corresponding parallel duplexes was also assessed using targeting to the purine strand ON18^{[36]} (Table 1).

The corresponding aryl imidazonaphthalimide analogues were synthesized according to scheme 3—here with phenyl imidazonaphthalimide as an example:

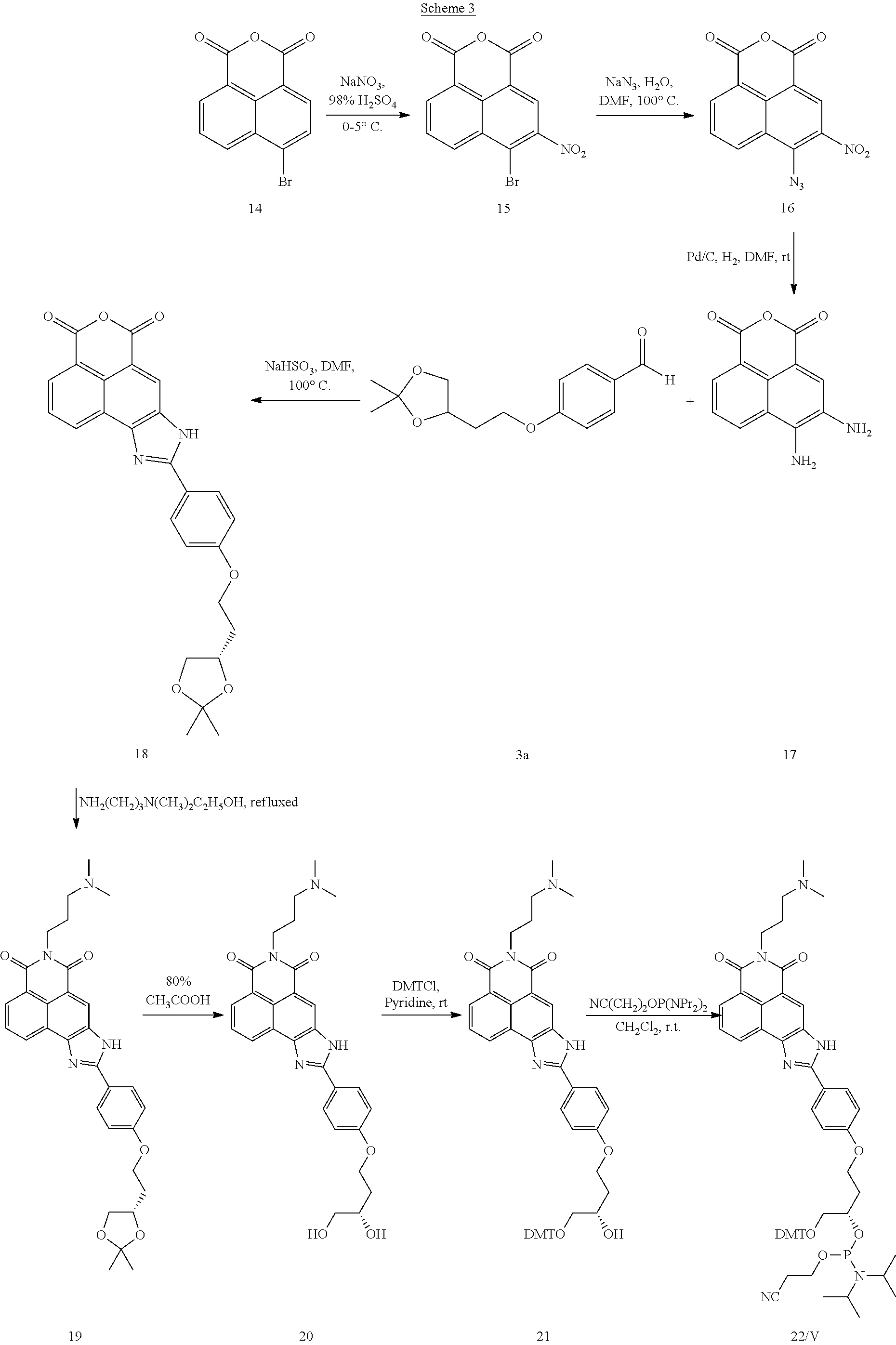

TABLE 1
Tₘ (° C.) data for triplex and duplex melting, evaluated from
UV melting curves (λ = 260 nm)

| | | Parallel triplex^{a} | | |
|---|---|---|---|---|
| | | 3′-CTGCCCCTTTCTTTTTT | | Parallel duplex^{b} |
| | | 5′-GACGGGGAAAGAAAAAA | | 5′-GACGGGGAAAGAAAAAA |
| | | (D1) | | (ON18) |
| Entry | TFO | pH 6.0 | pH 7.2 | pH 6.0 |
| ON1 | 5′-CCCCTTTCTTTTTT-3′ | 28.0 | <5.0 | 19.0 |
| ON2 | 5′-CCCCTTWTCTTTTTT-3′ | 45.5 | 28.0 | 33.5^{c} |
| ON3 | 5′-CCCCTTXTCTTTTTT-3′ | 46.5 | 26.0 | 31.5 |
| ON4 | 5′-CCCCTTYTCTTTTTT-3′ | 40.5 | 18.5 | 21.5 |
| ON5 | 5′-CCCCTTZTCTTTTTT-3′ | 10.5 | —^{d} | —^{d} |
| ON6 | 5′-CCCCTTTCWTTTTTT-3′ | 39.5^{c} | 21.5^{c} | 30.0^{c} |
| ON7 | 5′-CCCCTTTCXTTTTTT-3′ | 43.5 | 25.0 | 34.5 |
| ON8 | 5′-CCCCTTTCYTTTTTT-3′ | 35.5 | 18.5 | 23.0 |
| ON9 | 5′-CCCCTTTCZTTTTTT-3′ | 13.5 | —^{d} | —^{d} |
| ON10 | 5′-CCCCTTTCTXTTTTT-3′ | 48.5^{e} | 33.5 | 31.5 |
| ON11 | 5′-CCCCTTTCTYTTTTT-3′ | 38.5 | 18.5 | 19.5 |
| ON12 | 5′-CCCCTTWTCTWTTTTT-3′ | 56.5^{c,e} | 43.0^{c} | 38.0^{c} |
| ON13 | 5′-CCCCTTXTCTXTTTTT-3′ | 51.5^{e} | 37.0 | 37.5 |
| ON14 | 5′-CCCCTTYTCTYTTTTT-3′ | 46.5 | 15.0 | 20.5 |
| ON15 | 5′-WCCCCTTTCTTTTTT-3′ | 44.5^{c} | 20.5^{c} | 36.0^{c} |
| ON16 | 5′-XCCCCTTTCTTTTTT-3′ | 46.0 | 20.5 | 34.0 |
| ON17 | 5′-CCCCTTTCTTTTTTX-3′ | 43.5 | 20.0 | 31.5 |
| ON18 | 5′-CCCCTTTCVTTTTTT-3′ | 38.5 | | |

^{a}C = 1.5 μM of ON1-17 and 1.0 μM of each strand of dsDNA(D1) in 20 mM sodium cacodylate, 100 mM NaCl, 10 mM MgCl₂, pH 6.0 and 7.2. Duplex Tₘ = 58.5° C. (pH 6.0) and 57.0° C. (pH 7.2).
^{b}C = 1.0 μM of each strand in 20 mM sodium cacodylate, 100 mM NaCl, 10 mM MgCl₂, pH 6.0.
^{c}Data taken from Ref 28a.
^{d}Not determined.
^{e}Third strand and duplex melting overlaid. Tₘ values determined at 373 nm.

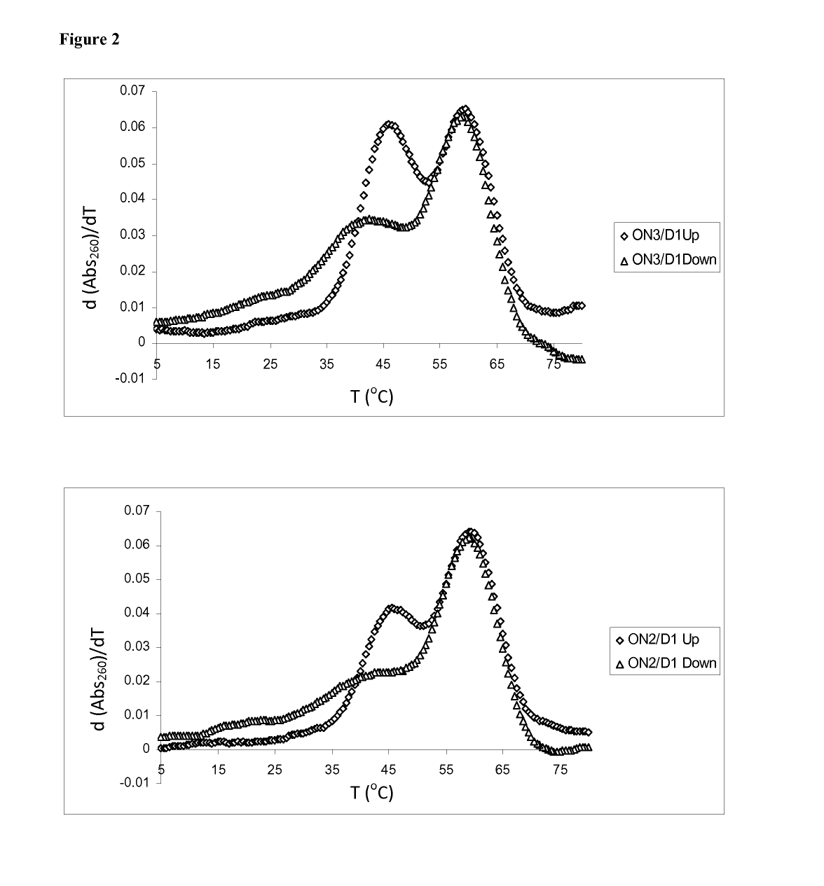

Stabilization of parallel triplexes was found in all cases when compared with the wild type ON1 at pH 6.0 and 7.2 except in case of ON5 and ON9 with insertion of the truncated intercalator Z. At pH 6.0 the stability of the modified sequences ON10 and ON13 with the intercalator X were also measured at a wavelength of λ=373 nm, because of overlapping curves at λ=260 nm for triplex and duplex melting. At pH 6 and independently of the site of insertion of the intercaltor X, the triplex stabilities of ON3/D1 (Tₘ=46.5° C.), ON7/D1 (Tₘ=43.5° C.) and ON10/D1 (Tₘ=48.5° C.) are enormously increased compared to the unmodified triplex ON1/D1 (Tₘ=28.0° C.). The observed stabilization in the range of ΔTₘ=15.5-20.5° C. corresponds to an excellent intercalating system. When thermal melting using the insertions of X in ON3 and ON7 is compared with W in ON2 and ON6 almost identical triples stabilities are observed at pH 6.0 and 7.2 although with a small preference of X over W in three out of four cases. The opposite trend is observed upon double insertions when on ON12/D1 is compared with ON13/D1. This may reflect that unwinding of the duplex for perfect stacking with the intercalator in a stringent triplex structure may be more difficult to achieve for two insertions. Another interesting difference between the intercalators W and X was observed in annealing experiments where X gave a more clear annealing temperature upon cooling a mixture of ON3 and D1 (FIG. 2).

The importance of a large aromatic ring system as an intercalator was confirmed by observing that the truncated intercalator Z inserted as a bulge gave less stable parallel triplexes (ON5 and ON9) when compared with the wild type ON1 and at pH 6.0. As discussed later on under molecular modeling, this confirms that the stability of the triplexes with bulge insertions of X is due to intercalation. Therefore, it was thought an advantage to replace the benzene ring in the intercalator X with the larger naphthalene ring to obtain the intercalator Y which was believed to give better stacking with the base pairs of the TFO. However, considerably lower triplex melting (6-15° C. at pH 6.0 and 7.2) was observed for the Y containing oligos ON4, ON8 and ON11 than for the X containing oligos ON3, ON7 and ON10, respectively. This is explained under molecular modeling by steric hindrance to planarity when naphthalene is incorporated into the intercalator. Attaching the intercalator X at the 5′-end (ON16) gave better stabilization of Hoogsteen-type triplexes and duplexes than at the 3′-end (ON17).

The parallel triplexes with bulge insertion of the intercalators W, X and Y in the middle of the TFO were studied for their sensitivity to Hoogsteen mismatches at pH 6.0 (Table 2). For mono insertions, X was slightly better than W to discriminate neighboring Hoogsteen mismatches in ON3 (15-23.5° C.) compared to ON2 (11-18.5° C.), respectively. For X, it is approximately the same range that is found for discrimination for a non-neighboring insertion (ON10). The worst case for discrimination was actually found when the study was extended to TFOs with double insertions of the intercalators X and Y separated by three nucleobases. Here the triplex containing ON13/D4 gave the smallest change in ΔTₘ=9.5° C. for replacement of a T/A base pair with a G/C base pair in the duplex part of the triplex. The discriminating power of a mono inserted intercalator should be compared with the work of Zhou et al^{[37]} who was actually aiming at stabilizing triplex forming of mismatch. They inserted 2-methoxy-6-chloro-9-aminoacridine in the middle of the TFOs as a bulge insertion and the ΔTₘ values were in the range of 10° C. which is a much lower discriminating power than the ones found for our intercalators.

If the ultimate goal is to use modified TFOs as antigene oligos to control diseases, it is also important to consider the effect of the modification if the oligo can make stable complexes with other targets, e.g. forming a parallel duplex by Hoogsteen bonding or normal antiparallel DNA/DNA or DNA/RNA duplexes. Here the TFOs were also targeted in a parallel duplex fashion to the oligo ON18. As it can be seen from Table 1 considerable stabilizations (12.5-15.5° C. at pH 6.0) are achieved for the intercalator X for mono insertions when compared with the wild type parallel duplex. This is slightly lower than the stabilizations (15.5-20.5° C. at pH 6.0) found for the corresponding triplexes. Besides, it is important to note that the triples melting is 9-17° C. higher than the corresponding parallel duplex melting.

TABLE 2
Tₘ (° C.) data for mismatched Hoogsteen parallel triplex^{a} melting,
evaluated from UV melting curves (λ = 260 nm) at pH 6.0

| | | Sequence | | | |
|---|---|---|---|---|---|
| | | 3′-CTGCCCCTTKCTTTTTT | | | |
| | | 5′-GACGGGGAALGAAAAAA | | | |
| | | D1, | D2, | D3, | D4, |
| Entry | TFO | K•L = T•A | K•L = A•T | K•L = C•G | K•L = G•C |
| ON1 | 5′-CCCCTTTCTTTTTT-3′ | 28.0 | <5.0 | <5.0 | <5.0 |
| ON2 | 5′-CCCCTTWTCTTTTTT-3′ | 45.5 | 27.0^{b} | 34.5^{b} | 28.5^{b} |
| ON3 | 5′-CCCCTTXTCTTTTTT-3′ | 46.5 | 23.0 | 29.5 | 31.5 |
| ON4 | 5′-CCCCTTYTCTTTTTT-3′ | 40.5 | 16.5 | 21.0 | 25.5 |
| ON10 | 5′-CCCCTTTCTXTTTTT-3′ | 48.5 | 30.5 | 33.0 | 35.5 |
| ON11 | 5′-CCCCTTTCTYTTTTT-3′ | 38.5 | 21.0 | 22.5 | 26.0 |
| ON13 | 5′-CCCCTTXTCTXTTTTT-3′ | 51.5 | 35.5 | 37.0 | 42.0 |
| ON14 | 5′-CCCCTTYTCTYTTTTT-3′ | 46.5 | 24.0 | 33.5 | 17.5 |

^{a}C = 1.5 μM of each oligonucleotide and 1.0 μM of each strand of dsDNA in 20 mM sodium cacodylate, 100 mM NaCl, 10 mM MgC1₂, pH 6.0.
^{b}Data taken from Ref 28a.

The thermal stability studies of antiparallel Hoogsteen-type DNA/DNA duplexes were observed at pH 6.0, pH 7.2 and the corresponding DNA/RNA duplex was performed at pH 7.0 (Table 3). As shown for ON2, ON6 and ON12, destabilization has been described for oligos including the intercalator W in the middle of the oligo towards ON19 in antiparallel Watson-Crick-type DNA/DNA duplexes, when compared with the wild type duplex.^{[28a]} Considering the similarity of W and X when used as conjugated bulge intercalators in triplex studies, it was surprising to find that the melting temperatures of both DNA/DNA and DNA/RNA duplexes with bulging X showed nearly identical melting temperatures to the corresponding wild type duplexes (ON3, ON7 and ON10). This holds even for double insertion of X (ON13). When the intercalators W and X were placed at the 5′-end in ON15, ON16, respectively or at the 3′-end in ON17, the stabilization effect was in the range ΔTₘ=3.5-7.0° C. for both DNA and RNA targeting. This is ascribed to stacking of the aromatic system on the adjacent nucleobases, which is known as the lid-effect.^{[38,39]}

TABLE 3
Tₘ (° C.) data for Watson-Crick antiparallel duplexes melting,
evaluated from UV melting curves (λ = 260 nm)

| | | DNA^{a} | | RNA^{b} |
|---|---|---|---|---|
| | | 3′-GGGGAAAGAAAAAA | | 3′-r(GGGGAAAGAAAAAA) |
| | | (ON19) | | (ON20) |
| Entry | Sequences | pH 6.0 | pH 7.2 | pH 7.0 |
| ON1 | 5′-CCCCTTTCTTTTTT-3′ | 49.5 | 49.5 | 52.0 |
| ON2 | 5′-CCCCTTWTCTTTTTT-3′ | 46.5^{c} | 45.5^{c} | —^{d} |
| ON3 | 5′-CCCCTTXTCTTTTTT-3′ | 50.5 | 50.5 | 53.0 |
| ON4 | 5′-CCCCTTYTCTTTTTT-3′ | 46.5 | 46.0 | 49.5 |
| ON6 | 5′-CCCCTTTCWTTTTTT-3′ | 44.5 | —^{d} | —^{d} |
| ON7 | 5′-CCCCTTTCXTTTTTT-3′ | 51.0 | 50.5 | 51.0 |
| ON8 | 5′-CCCCTTTCYTTTTTT-3′ | 46.0 | 46.0 | 49.0 |
| ON10 | 5′-CCCCTTTCTXTTTTT-3′ | 51.0 | 51.0 | 53.0 |
| ON11 | 5′-CCCCTTTCTYTTTTT-3′ | 47.5 | 47.5 | 49.5 |
| ON12 | 5′-CCCCTTWTCTWTTTTT-3′ | 41.0^{c} | 38.0^{c} | —^{d} |
| ON13 | 5′-CCCCTTXTCTXTTTTT-3′ | 49.0 | 50.5 | 49.5 |
| ON14 | 5′-CCCCTTYTCTYTTTTT-3′ | 38.5 | 38.5 | 42.5 |
| ON15 | 5′-WCCCCTTTCTTTTTT-3′ | 53.0^{c} | 52.0^{c} | —^{d} |
| ON16 | 5′-XCCCCTTTCTTTTTT-3′ | 56.5 | 56.5 | 59.0 |
| ON17 | 5′-CCCCTTTCTTTTTTX-3′ | 54.0 | 54.0 | 55.5 |

^{a}C = 1.0 μM of each oligonucleotide in 20 mM sodium cacodylate, 100 mM NaCl, 10 mM MgCl₂, pH 6.0 and 7.2.
^{b}C = 1.0 μM of each oligonucleotide in 140 mM NaCl, 10 mM sodium phosphate buffer, 1 mM EDTA, pH = 7.0.
^{c}Data taken from Ref 28a.
dNot determined.

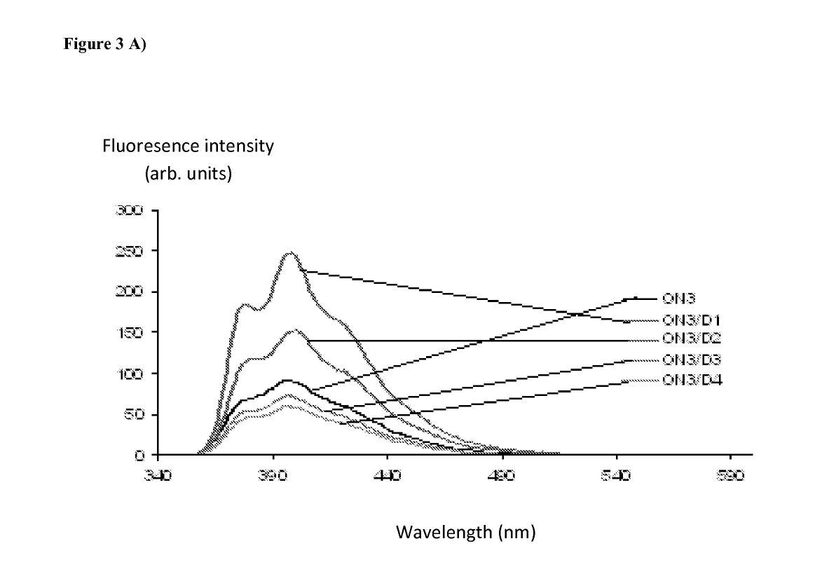

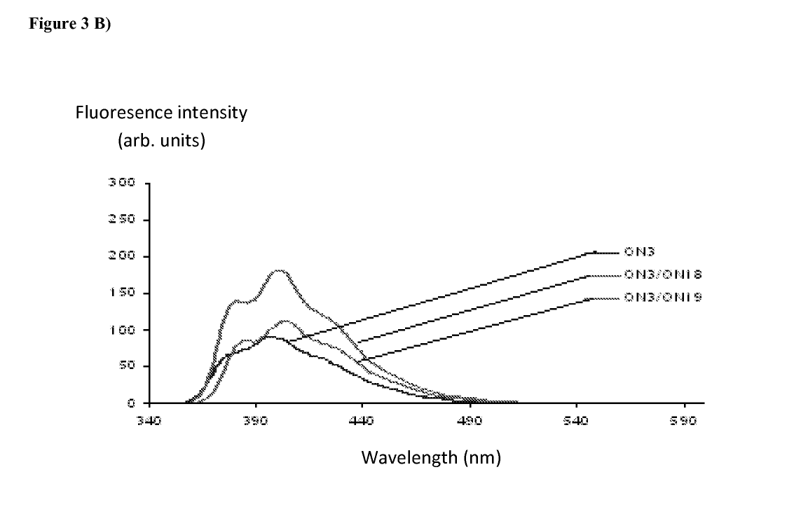

The fluorescence measurements were performed for the single strand TFO (ON3) which was found effective to form triplexes and to discriminate Hoogsteen mismatches. The insertion of the intercalator X into oligonucleotides resulted in a characteristic monomeric fluorescence spectrum, with maxima at 400 nm upon excitation at 373 nm (FIG. 3). In all cases, a 4 nm shift of monomeric fluorescence was detected upon formation of triplexes or duplexes except in two cases ON3/D3, ON3/D4. The spectra were recorded from 340 nm to 600 nm at 10° C. in the same buffer solutions use for Tₘ studies using a 1.0 μM concentration of each strand of the unmodified duplex and modified TFO for the duplex and triplex measurements. Excitation and emission slits were set to 4 nm and 0.0 nm, respectively. The fluorescence spectra of the TFO ON3 towards D1, D2, D3 and D4 were recorded at pH 6.0 and they are shown in FIG. 3A. The fluorescence intensity increased of the fully matched triplex ON3/D1 compared to the single-stranded ON3. However, the emission intensity of the triplex Hoogsteen mismatched ON3/D2 decreased slightly because of an inverted A/T base pair in the duplex next to the intercalator compared to the matching triplex, On the contrary, when a Hoogsteen mismatch was due to a C/G base pair near the insertion of the intercalating X (ON3/D3, ON3/D4), the fluorescence intensity was even lower than the one of the single strand TFO. The fluorescence spectra of the oligo ON3 towards ON18, ON19 in parallel and antiparallel duplexes, respectively, are shown in FIG. 3B. The emission intensity of the antiparallel duplex ON3/ON19 is comparable to the one of the single strand ON3 where as the parallel duplex ON3/ON18 showed increased fluorescence intensity.

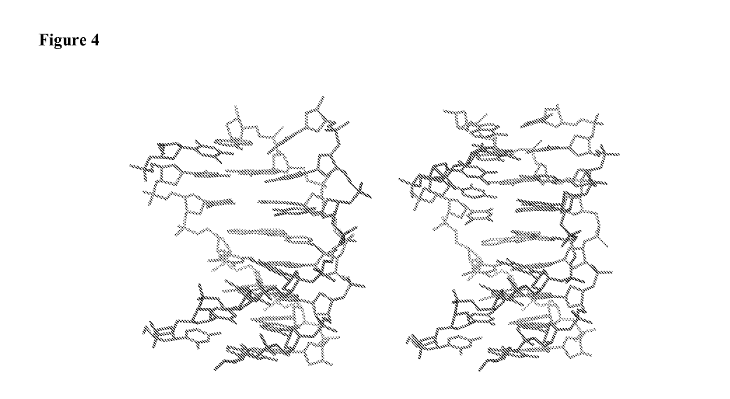

The novel monomers X and Ys ability to stabilize the triplex via intercalation were studied using representative low-energy structures generated with the AMBER* force field in MacroModel 9.1. Molecular modeling was performed on truncated triplexes with the intercalator incorporated into the middle of the triplex. As it can be seen from FIG. 4, the position of the intercalators, X and Y, are similar and in both cases are the phenanthroimidazole-moiety positioned in the Watson-Crick duplex thereby adding to the triplex stability via π-π-interaction. In addition, the phenyl- and naphthalene-moiety are positioned between nucleobases of the TFO, adding to the stability as well as insuring equal amount of unwinding at the site of intercalation. In the case of intercalator X, the phenyl-moiety is only slightly twisted in comparison to the naphthalene-moiety of intercalator Y which is forced out of plane by sterical interaction between protons on the naphthalene-moiety and on the imidazole-moiety. The large extent of twisting between the two aromatic moieties of Y forces the nucleobases of the TFO to twist out of plane compared to X, thereby weakening the Hoogsteen hydrogen bonds. This conclusion supports the thermal stability measurements which showed a decrease in triplex stability using intercalator Y in comparison with intercalator X, clearly demonstrates the importance of optimal Hoogsteen hydrogen-bonds and π-π-interactions.

Twisting the naphthalene-moiety of intercalator Y 180° around the single bond resulted in almost identical interacting properties of the intercalator with the triplex and no optimal conformation could be assigned.

Here we have described the synthesis of two intercalating nucleic acid monomers X and Y, and their incorporation into oligonucleotides giving in good yield using normal oligonucleotide synthesis procedures. Melting studies showed that the two intercalators have extraordinary high thermal stability of Hoogsteen-type triplexes and duplexes with a high discrimination of mismatch strands. DNA-strands containing intercalator X show higher thermal triplex stability than DNA-strands containing intercalator Y. Interestingly, when inserted the intercalator X (ON7) showed increased the triplex stability than the intercalator W (TINA). The linker must be chosen in unity with the intercalator, even though a five atom linker seems like the optimal length for bulge insertions in a DNA duplex. In our research, the linker was the same atom number of the previous studies (TINA) but differs in that the oxygen atom was attached directly to the phenyl or naphthyl rings, respectively. The introduction of a fused imidazol ring can lead to the formation of a larger aromatic system and consequently to a higher affinity for the DNA molecular, and must have an effect on the electrostatic properties of the chromophore. Larger intercalating phenanthroimidazol moiety was an advantage for triplex stabilization. This work was confirmed by the synthesis of intercalator Z which gave less stable parallel triplexes, when inserted as a bulge which means that imidazol ring did not stack with any of the bases in the triplex structure.

### EXAMPLES

NMR spectra were recorded on a Varian Gemini 2000 spectrometer at 300 MHz for ¹H, 75 MHz for ¹³C and 121.5 MHz for ³¹P with TMS as an internal standard for ¹H NMR, deuterated solvents CDCl₃ (δ 77.00 ppm), DMSO-d₆ (δ 39.44 ppm) for ¹³C NMR, and 85% H₃PO₄ as an external standard for ³¹P NMR. MALDI mass spectra of the synthesized compounds were recorded on a Fourier Transform Ion Cyclotron Resonance Mass Spectrometer (IonSpec, Irvine, Calif.). For accurate ion mass determinations, the (MH⁺) or (MNa⁺) ion was peak matched using ions derived from the 2,5-dihydroxybenzoic acid matrix. Electrospray ionization mass spectra (ESI-MS) were performed on a 4.7 T HiResESI Uitima (FT) mass spectrometer. Both spectrometers are controlled by the OMEGA Data System. Melting points were determined on a Büchi melting point apparatus. Silica gel (0.040-0.063 mm) used for column chromatography and analytical silica gel TLC plates 60 F₂₅₄ were purchased from Merck. Solvents used for column chromatography were distilled prior to use, while reagents were used as purchased. Petroleum ether (PE): by 60-80° C.

### Example 1

General procedure for preparation of 3 in a Mitsunobu reaction. An ice-cooled solution of diethylazodicarboxylate (DEAD, 2.5 ml, 16 mmol) in dry THF (155 ml) was treated with (S)-2-(2,2-dimethyl-1,3-dioxolan-4-yl)ethanol (1) (1.9 ml, 13 mmol) for 25 min, and then 4-hydroxybenzaldehyde (2a) (2.1 g, 17 mmol) or 4-hydroxy-1-naphthaldehyde (2b) (3.0 g, 17 mmol) and triphenylphosphine (4.2 g, 16 mmol) were added to the mixture. The mixture was stirred in an ice water bath for 30 min, and then allowed to warm to room temperature overnight. The mixture was quenched with aqueous ammonia (105 ml) and extracted with AcOEt. The organic layer was washed with water, dried over MgSO₄, and concentrated under reduced pressure to leave an oil which was purified by silica gel column chromatography [petroleum ether/diethyl ether (1:1, v/v)] to afford the pure products 3a,b.

(S)-4-(2-(2,2-Dimethyl-1,3-dioxolan-4-yl)ethoxy)benzaldehyde (3a). Yield: 3.5 g (81%) as an oil; R_{f} 0.30 (50% petroleum ether/diethyl ether). ¹H NMR (CDCl₃): δ 1.38 (s, 3H, CH₃), 1.44 (s, 3H, CH₃), 2.08 (m, 2H, CH₂CH₂O), 3.67 (m, 1H, CHH), 4.12-4.22 (m, 3H, CHH and CH₂CH₂O), 4.32 (m, 1H, CH), 7.01 (d, 2H, J=8.7 Hz, aryl), 7.84 (d, 2H, J=8.7 Hz, aryl), 9.88 (s, 1H, CHO). ¹³C NMR (CDCl₃): δ 25.6 (CH₃), 26.9 (CH₃), 33.3 (CH₂CH₂O), 69.4 (CH₂OC(CH₃)₂), 73.0 (CH₂CHCH₂), 108.9 (C(CH₃)₂), 114.6, 130.0, 131.9, 163.8 (aryl), 190.7 (CHO). HRMS (ESI) m/z Calcd for C₁₄H₁₈O₄Na⁺ (MNa⁺) 273.1097 Found 273.1101.

(S)-4-(2-(2,2-Dimethyl-1,3-dioxolan-4-yl)ethoxy)-1-naphthaldehyde (3b). Yield 4.8 g (92%) as an oil; R_{f} 0.31 (50% petroleum ether/diethyl ether). ¹H NMR (CDCl₃): δ 1.39 (s, 3H, CH₃), 1.44 (s, 3H, CH₃), 2.23 (m, 2H, CH₂CH₂O), 3.74 (dd, 1H, J=7.2, 8.1 Hz, CHH), 4.21 (m, 1H,CH), 4.39 (m, 3H, CH₂CH₂O, CHH), 6.93 (d, 1H, J=8.1 Hz, aryl), 7.57-7.60 (m, 1H, aryl), 7.68-7.71 (m, 1H, aryl), 7.90 (d, 1H, J=8.1 Hz, aryl), 8.31 (d, 1H, J=9.0 Hz, aryl), 9.31(d, 1H, J=9.0 Hz, aryl), 10.20 (s, 1H, CHO). ¹³C NMR (CDCl₃): δ 25.7 (CH₃), 27.0 (CH₃), 33.4 (CH₂CH₂O), 65.5 (CH₂CH₂O), 69.5 (CH₂OC(CH₃)₂), 73.2 (CH₂CHCH₂), 103.6 (aryl), 109.1 (C(CH₃)₂), 122.2, 124.9, 125.0, 125.4, 126.7, 129.5, 131.9, 139.6, 159.9 (aryl), 192.2 (CHO). HRMS (ESI) m/z Calcd for C₁₈H₂₀O₄Na⁺ (MNa⁺) 323.1254 Found 323.1264.

### Example 2

General procedure for preparation of the phenanthroimidazol compounds 6. Phenanthrene-9,10-dione (1 equiv.) and ammonium acetate (16.5 equiv.) were dissolved in hot glacial acetic acid (10 ml). While the mixture was stirred, a solution of (S)-4-(2-(2,2-dimethyl-1,3-dioxolan-4-yl)ethoxy)benzaldehyde (3a, 2.0 g, 8.0 mmol) or (S)-4-(2-(2,2-dimethyl-1,3-dioxolan-4-yl)ethoxy)-1-naphthaldehyde (3b, 1.0 g, 3.3 mmol) in 10 ml of glacial acetic acid was added dropwise. The mixture was heated at 90° C. for 3 h and was then poured in to water (200 ml). The mixture was neutralized with aqueous ammonia to pH 7 and then cooled to room temperature. The precipitate was filtered off and washed with large portions of H₂O. The residue was purified by silica gel column chromatography [MeOH/CH₂Cl₂ (1:1, v/v)] afforded 5 and 6a. Compound 6b was obtained directly from the precipitate without using chromatography. Recrystallization from toluene and one drop of NEt₃.

(S)-2-(4-(2-(2,2-Dimethyl-1,3-dioxolan-4-yl)ethoxy)phenyl)-1H-phenanthro[9,10-d]imidazole (5). Yield 0.30 g (8.5%) as solid; R_{f} 0.55 (50% MeOH/CH₂Cl₂); mp 196-198° C. ¹H NMR (CDCl₃): δ 1.35 (s, 3H, CH₃), 1.41 (s, 3H, CH₃), 1.91 (m, 2H, CH₂CH₂O), 3.55 (m, 1H, CHH), 3.82 (m, 2H, CHH, CH₂CHHO), 4.05 (m, 1H, CH₂CHHO), 4.18 (m, 1H, CH), 6.64 (d, 2H, J=8.7 Hz, aryl), 7.54 (m, 4H, aryl), 7.89 (d, 2H, J=8.7 Hz, aryl), 8.43 (br s, 2H, aryl), 8.67 (m, 2H, aryl). ¹³C NMR (CDCl₃): δ 25.7 (CH₃), 26.9 (CH₃), 33.3 (CH₂CH₂O), 64.5 (CH₂CH₂O), 69.5 (CH₂OC(CH₃)₂), 73.3 (CH₂CHCH₂), 108.8 (C(CH₃)₂), 114.5, 121.7, 122.7-128.2 (aryl), 149.35 (C═N, aryl), 159.7 (aryl). HRMS (MALDI) m/z Calcd for C₂₈H₂₇N₂O₃⁺ (MH⁺) 439.2016 Found 439.2002.

(S)-4-(4-(1H-Phenanthro[9,10-d]imidazol-2-yl)phenoxy)butane-1,2-diol (6a). Yield 2.3 g (72%) as solid; R_{f} 0.10 (50% MeOH/CH₂Cl₂); mp 263-265° C. ¹H NMR (DMSO-d₆): δ 1.77 (m, 1H, CHHCH₂O), 2.04 (m, 1H, CHHCH₂O), 3.42 (m, 2H, CHHOH and CHOH), 3.76 (m, 1H, CHHOH), 4.23 (m, 2H,CH₂CH₂O), 4.69, 4.76 (2s, 2H, 2×OH), 7.20 (d, 2H, J=8.7 Hz, aryl), 7.63 (m, 2H, aryl), 7.75 (m, 2H, aryl), 8.30 (d, 2H, J=8.7 Hz, aryl), 8.61 (d, 2H, J=8.1 Hz, aryl), 8.83 (d, 2H, J=8.1 Hz, aryl), 13.32 (br s, 1H, NH). ¹³C NMR (DMSO-d₆): δ 33.1 (CH₂CH₂O), 64.8 (CH₂CH₂O), 66.0 (CHCH₂OH), 68.1 (CHCH₂OH), 114.8, 121.9, 122.8-127.7 (aryl), 149.4 (C═N, aryl), 159.7 (aryl). HRMS (MALDI) m/z Calcd for C₂₅H₂₃N₂O₃⁺ (MH⁺) 399.1703 Found 399.1689.

(S)-4-(4-(1H-Phenanthro[9,10-d]imidazol-2-yl)naphalen-1-yloxy)butane-1,2-diol (6b). Yield 1.2 g (80%) as solid; mp 165-167° C. ¹H NMR (DMSO-d₆): δ 2.05 (m, 2H, CH₂CH₂O), 3.61 (m, 1H, CHOH), 3.85 (m, 1H, CHHOH), 4.06 (m, 1H, CHHOH), 4.41 (m, 2H, CH₂O), 4.73, 5.16 (2br s, 2H, 2×OH), 7.23 (d, 1H, J=7.8 Hz, aryl), 7.61-7.78 (m, 7H, aryl), 8.09 (d, 1H, J=8.1 Hz, aryl), 8.36 (d, 1H, J=7.8 Hz, aryl), 8.61 (m, 1H, aryl), 8.88 (m, 2H, aryl), 9.24 (d, 1H, J=8.1 Hz, aryl), 13.49 (br s, 1H, NH). ¹³C NMR (DMSO-d₆): δ 33.1 (CH₂CH₂O), 65.2 (CH₂CH₂O), 66.1 (CHCH₂OH), 68.2 (CHCH₂OH), 104.6, 120.0, 121.9, 122.0-131.7 (aryl), 149.6 (C═N, aryl), 155.3 (aryl). HRMS (ESI) m/z Calcd for C₂₉H₂₅N₂O₃⁺ (MH⁺) 449.1860 Found 449.1864.

### Example 3

General procedure for preparation of 7 by DMT-protection. (S)-4-(4-(1H-Phenanthro[9,10-d]imidazol-2-yl)phenoxy)butane-1,2-diol (6a, 1.0 g, 2.5 mmol) or (S)-4-(4-(1H-phenanthro[9,10-d]imidazol-2-yl)naphalen-1-yloxy)butane-1,2-diol (6b, 0.50 g, 1.11 mmol) was dissolved in anhydrous pyridine (20 ml). 4,4′-Dimethoxytrityl chloride (1.2 equiv.) was added under a nitrogen atmosphere, and the reaction mixture was stirred at room temperature for 24 h. The reaction was quenched by addition of MeOH (2 ml) followed by addition of EtOAc (75 ml), and extracted with saturated aqueous NaHCO₃ (2×20 ml). The H₂O phase was extracted with EtOAc (2×10 ml), and the combined organic phases were dried (Na₂SO₄), filtered, and evaporated under diminished pressure. The residue was coevaporated twice with toluene/EtOH 15 ml, (1:1, v/v). The residue was purified by silica gel column chromatography [NEt₃ (0.5%, v/v)/EtOAc (40-50%)/cyclohexane] to afford the DMT-protected diols 7a,b.

(S)-4-(4-(1H-Phenanthro[9,10-d]imidazol-2-yl)phenoxy)-1-(bis(4-methoxyphenyl)(phenyl)methoxy)butan-2-ol (7a). Yield 1.4 g (79%) as a foam; R_{f} 0.43. ¹H NMR (CDCl₃): δ 1.85 (m, 2H, CH₂CH₂O), 3.18 (m, 2H, CH₂ODMT), 3.72 (s, 6H, 2×OCH₃), 3.89 (m, 2H, CH₂CH₂O), 4.04 (m, 1H, CHOH), 6.66 (d, 2H, J=8.4 Hz, aryl), 6.77 (d, 4H, J=8.7 Hz, DMT), 7.17-7.30 (m, 9H, aryl), 7.40 (d, 2H, J=7.2 Hz, aryl), 7.55 (m, 4H, aryl), 7.88 (d, 2H, J=8.4 Hz, aryl), 8.44 (br s, 1H, NH), 8.69 (m, 2H, aryl). ¹³C NMR (CDCl₃): δ 33.0 (CH₂CH₂O), 55.2 (2×OCH₃), 64.7 (CH₂CH₂O), 67.4 (CHOH), 68.4 (CH₂ODMT), 86.2 (OCPh₃), 113.1, 114.7, 122.7-130.0, 135.9, 144.8, 149.6, 158.5, 159.7 (aryl). HRMS (ESI) m/z Calcd for C₄₆H₄₁N₂O₅⁻ (MH⁺) 701.3010 Found 701.3044.

(S)-4-(4-(1H-Phenanthro[9,10-d]imidazol-2-yl)naphthalen-1-yloxy)-1-(bis(4-methoxy phenyl)(phenyl)methoxy)butan-2-ol (7b). Yield 0.47 g (56%) as a foam; R_{f} 0.34. ¹H NMR (CDCl₃): δ 1.90 (m, 2H, CH₂CH₂O), 3.02 (br s, 1H, OH), 3.18 (m, 2H, CH₂ODMT), 3.75 (s, 6H, 2×OCH₃), 3.93 (m, 2H, CH₂CH₂O), 4.07 (m, 1H, CHOH), 6.33 (m, 1H, aryl), 7.76 (d, 4H, J=8.4 Hz, DMT), 7.18-7.55 (m, 18H, aryl), 8.04 (d, 1H, J=7.5 Hz, aryl), 8.55 (d, 1H, J=7.5 Hz, aryl), 8.69 (m, 2H, aryl), 11.31 (br s, 1H, NH). ¹³C NMR (CDCl₃): δ 33.1 (CH₂CH₂O), 55.2, 55.2 (2×OCH₃), 64.8 (CH₂ CH₂O), 67.5 (CHOH), 68.5 (CH₂ODMT), 86.2 (OCPh₃), 103.7, 113.1, 120.2, 122.0, 125.1-130.0, 132.1, 135.9, 144.8 (aryl), 149.5 (C═N, aryl), 155.5, 158.4 (aryl). HRMS (ESI) m/z Calcd for C₅₀H₄₂N₂O₅Na⁺ (MNa⁺) 773.2987 Found 773.3003.

### Example 4

General procedure for preparation of phosphoramidite 8. DMT-protected compound 7a (0.4 g, 0.57 mmol) or 7b (0.1 g, 0.17 mmol) was dissolved under an argon atmosphere in anhydrous CH₂Cl₂ (10-15 ml). N,N′-Diisopropylammonium tetrazolide (1.5 equiv.) was added, followed by dropwise addition of 2-cyanoethyl N,N,N′,N′-tetraisopropylphosphordiamidite (3 equiv.) under external cooling with an ice-water bath. The reaction mixture was stirred at room temperature overnight. After 24 h, analytical TLC showed no more starting material and the reaction was quenched with H₂O (10-20 ml). The layers were separated and the organic phase was washed with H₂O (10-20 ml), the combined water layers were washed with CH₂Cl₂ (25 ml), the organic phase was dried (Na₂SO₄) and filtered, and the solvents were evaporated in vacuo. The residue was purified by silica gel column chromatography [NEt₃ (0.5%, v/v)/EtOAc (40-50%)/cyclohexane] to afford the final products 8a,b as a foam, which were used in DNA synthesis after drying under diminished pressure.

(S)-4-(4-(1H-Phenanthro[9,10-d]imidazol-2-yl)phenoxy)-1-(bis(4-methoxyphenyl)(phenyl)-methoxy)butan-2-yl 2-cyanoethyl diisopropylphosphoramidite (8a). Yield 0.44 g (86%) as a foam; R_{f} 0.68. ¹³C NMR (CDCl₃): δ 20.1 (CH₂CN), 24.4, 24.5, 24.6, 24.7 (2×CH(CH₃)₂), 33.0 (CH₂CH₂O), 43.1, 43.2 (2×C(CH₃)₂), 55.2 (2×OCH₃), 57.8 (OCH₂CH₂CN), 64.1 (CH₂CH₂O), 66.4 (CHOP [NPr₂]₂), 69.4 (CH₂ODMT), 86.0 (OCPh₃), 113.0, 114.9, 122.5-130.1, 136.1, 136.2, 144.9, 149.8, 158.4, 158.4, 160.0 (aryl). ³¹P NMR (CDCl₃): δ 149.98, 150.05 in a 5:4 ratio. HRMS (ESI) m/z Calcd for C₅₅H₅₇N₄O₆PNa⁺ (MNa⁺) 923.3909 Found 923.3913.

(S)-4-(4-(1H-Phenanthro[9,10-d]imidazol-2-yl)naphthalen-1-yloxy)-1-(bis(4-methoxy phenyl)(phenyl)methoxy)butan-2-yl 2-cyanoethyl diisopropylphosphoramidite (8b). Yield 0.11 g (81%) as a foam; R_{f} 0.64. ¹³C NMR (CDCl₃): δ 20.08 (CH₂CN), 24.4, 24.5, 24.6, 24.7 (2×CH(CH₃)₂), 33.0 (CH₂CH₂O), 43.1, 43.3 (2×CH(CH₃)₂), 55.2 (2×OCH₃), 57.9 (OCH₂CH₂CN), 64.2 (CH₂CH₂O), 66.4 (CHOP[NPr₂]₂), 70.8 (CH₂ODMT), 86.1 (OCPh₃), 104.0, 113.1, 117.7, 120.6-132.5, 136.1, 136.2, 145.0, 149.5, 155.8, 158.4 (aryl). ³¹P NMR (CDCl₃): δ 149.98, 150.48 in a 2:1 ratio. HRMS (ESI) m/z Calcd for C₅₉H₅₉N₄O₆PNa⁺ (MNa⁺) 973.4065 Found 973.4021.

### Example 5

(S)-4-(3,4-Dihydroxybutoxy)-1-naphthaldehyde (9). Compound 3b (0.85 g, 2.83 mmol) was stirred in 80% acetic acid (25 ml) for 24 h at room temperature. The solvent was removed in vacuo, and the residue was coevaporated twice with toluene/EtOH (30 ml, 5:1, v/v). The residue was dried in vacuo to afford 4-(3,4-dihydroxybutoxy)-1-naphthaldehyde 9. Yield 0.74 g (100%) as an oil which was used in the next step without further purification. ¹H NMR (DMSO-d₆): δ 1.83 (m, 1H, CHHCH₂O), 2.30 (m, 1H, CHHCH₂O), 3.42 (m, 2H, CH₂CHOH, CHHOH), 3.80 (m, 1H, CHHOH), 4.42 (m, 2H, CH₂CH₂O), 4.63, 4.73 (s, 2H, 2×OH), 7.22 (m, 1H, aryl), 7.64 (m, 1H, aryl), 7.75 (m, 1H, aryl), 8.14 (d, 1H, J=8.1 Hz, aryl), 8.31 (d, 1H, J=7.8 Hz, aryl), 9.23 (d, 1H, J=8.4 Hz, aryl), 10.18 (s, 1H, CHO). ¹³C NMR (DMSO-d₆): δ 32.8 (CH₂CH₂O), 65.7 (CH₂CH₂O), 65.9 (CH₂OH), 68.0 (CHOH), 104.6, 122.1-131.1, 140.4, 159.6 (aryl), 192.7 (CHO). HRMS (ESI) m/z Calcd for C₁₅H₁₆O₄Na⁺ (MNa⁺) 283.0941 Found 283.0948.

### Example 6

(S)-4-(4-(1H-Imidazol-2-yl)naphthalen-1-yloxy)butan-1,2-diol (10). To a solution of (S)-4-(3,4-dihydroxybutoxy)-1-naphthaldehyde (9, 0.10 g, 0.38 mmol) in EtOH (0.54 ml) was added about dry MeCN (3 ml) to give a clear solution. 40% Glyoxal in H₂O (0.10 ml, 1.93 mmol) and 20 M ammonium hydroxide (0.13 ml) was added at 0° C. The mixture was stirred for 30 min at 0° C. and then at room temperature overnight. The mixture was concentrated in vacuo and the residue was purified by silica gel column chromatography [EtOAc/cyclohexane/NEt₃ (90:8:2, v/v/v)] to give compound 10. Yield 0.05 g (44%) as an oil; R_{f} 0.11. ¹H NMR (DMSO-d₆): δ 2.04 (m, 2H, CH₂CH₂O), 3.42 (m, 2H, CHOH and CHHOH), 3.80 (m, 1H, CHHOH), 4.36 (m, 2H, CH₂CH₂O), 4.69, 4.71 (2s, 2H, 2×OH), 6.70-8.01 (m, 6H, aryl), 8.27 (d, 1H, J=8.7 Hz, aryl), 9.01 (d, 1H, J=8.7 Hz, aryl), 12.38 (br s, 1H, NH). ¹³C NMR (DMSO-d₆): δ 33.1 (CH₂CH₂O), 65.0 (CH₂CH₂O), 66.0 (CH₂OH), 68.1 (CHOH), 104.2, 120.4, 121.5, 125.4, 126.8, 128.1, 129.8, 131.2, 134.8, 145.4, 154.3 (aryl). HRMS (MALDI) m/z Calcd for C₁₇H₁₈N₂O₃Na⁻ (MNa⁺) 321.1210 Found 321.1217.

### Example 7

(S)-4-(4-(Bis(4-methoxyphenyl)(phenyl)methoxy)-3-hydroxybutoxy)-1-naphthaldehyde (11). Compound 9 (0.50 g, 1.92 mmol) was dissolved in dry pyridine (20 ml) and 4,4′-dimethoxytrityl chloride (DMT-Cl) (0.78 g, 2.30 mmol) was added under a nitrogen atmosphere. The reaction mixture was stirred for 24 h at room temperature. The solvent was evaporated off under reduced pressure, and the residue was purified by silica gel column chromatography [NEt₃ (0.5%, v/v)/EtOAc (30-50%)/cyclohexane] affording compound 11. Yield 0.65 g (60%) as a foam; R_{f} 0.21. ¹H NMR (CDCl₃): δ 2.08 (m, 2H, CH₂CH₂O), 2.49 (s, 1H, OH), 3.21, 3.32 (2×m, 2H, CH₂ODMT), 3.76 (s, 6H, 2×OCH₃), 4.13 (m, 1H, CHOH), 4.34 (m, 2H, CH₂CH₂O), 6.80 (d, 4H, J=9.0 Hz, DMT), 6.86 (d, 1H, J=8.1 Hz, aryl), 7.29 (m, 8H, aryl), 7.43 (d, 1H, J=6.9 Hz, aryl), 7.56 (m, 1H, aryl), 7.72 (m, 1H, aryl), 7.89 (d, 1H, J=8.1 Hz, aryl), 8.22 (d, 1H, J=8.4 Hz, aryl), 9.30 (d, 1H, J=8.4 Hz, aryl), 10.19 (s, 1H, CHO). ¹³C NMR (CDCl₃): δ 32.9 (CH₂CH₂O), 55.2 (2×OCH₃), 65.3 (CH₂CH₂O), 67.4 (CHOH), 68.2 (CH₂ODMT), 86.3 (OCPh₃), 103.7, 113.2, 122.3-130.0, 131.9, 135.8, 139.7, 144.7, 158.5, 160.0 (aryl), 192.3 (CHO). HRMS (ESI) m/z Calcd for C₃₆H₃₄O₆Na⁺ (MNa⁺) 585.2248 Found 585.2253.

### Example 8

(S)-4-(4-(1H-Imidazol-2-yl)naphthalen-1-yloxy)-1-(bis(4-methoxyphenyl)(phenyl)-methoxy)butan-2-ol (12). To a solution of (S)-4-(4-(bis(4-methoxyphenyl)(phenyl)methoxy)-3-hydroxybutoxy)-1-naphthaldehyde (11) (0.44 g, 0.79 mmol) in EtOH (1.1 ml) was added dry MeCN (5 ml) to give a clear solution. 40% Glyoxal in H₂O (0.18 ml, 4.0 mmol) and 20 M ammonium hydroxide (0.27 ml) was added at 0° C. The mixture was stirred for 30 min at 0° C. and then at room temperature under a nitrogen atmosphere overnight. The reaction mixture was concentrated in vacuo and the residue was purified by silica gel column chromatography [EtOAc/cyclohexane/NEt₃ (90:8:2, v/v/v)] affording compound 12. Yield 0.15 g (32%) as a foam; R_{f}0.50. ¹H NMR (CDCl₃): δ 1.94 (m, 2H, CH₂CH₂O), 3.19, 3.29 (2×m, 2H, CH₂ODMT), 3.74 (s, 6H, 2×OCH₃), 4.11 (m, 4H, CH₂CH₂O and CHOH), 6.55 (d, 1H, J=8.1 Hz, aryl), 6.78 (d, 4H, J=8.7 Hz, DMT), 7.08 (s, 2H, imidazole), 7.19-7.31 (m, 7H, aryl), 7.41 (m, 5H, aryl), 8.16 (d, 1H, J=9.0 Hz, aryl), 8.44 (d, 1H, J=8.1 Hz, aryl). ¹³C NMR (CDCl₃): δ 33.1 (CH₂CH₂O), 55.2 (2×OCH₃), 64.8 (CH₂CH₂O), 67.5 (CHOH), 68.4 (CH₂ODMT), 86.2 (OCPh₃), 103.8, 113.1, 120.7, 122.1, 125.4-130.0, 132.0, 135.9, 136.0, 144.8, 146.4, 155.2, 158.4 (aryl). HRMS (ESI) m/z Calcd for C₃₈H₃₆N₂O₅Na⁺ (MNa⁺) 623.2517 Found 623.2494.

### Example 9

(S)-4-(4-(1H-Imidazol-2-yl)naphthalen-1-yloxy)-1-(bis(4-methoxyphenyl)(phenyl)-methoxy)butan-2-yl 2-cyanoethyl diisopropylphosphoramidite (13). Compound 12 (0.10 g, 0.17 mmol) was dissolved under an argon atmosphere in anhydrous CH₂Cl₂ (10 ml). N,N′-Diisopropyl ammonium tetrazolide (0.04 g, 0.25 mmol) was added, followed by dropwise addition of 2-cyanoethyl tetraisopropylphosphordiamidite (0.15 g, 0.45 mmol) under external cooling with an ice-water bath. The reaction mixture was stirred at room temperature under an argon atmosphere overnight. After 24 h, analytical TLC showed no more starting material. The solvent was evaporated under reduced pressure and the residue was purified by silica gel column chromatography [EtOAc/cyclohexane/NEt₃ (90:8:2, v/v/v)] affording compound 13. Yield: 0.11 g (81%) as a foam; R_{f} 0.70. ¹³C NMR (CDCl₃): δ 20.2 (CH₂CN), 24.4, 24.5, 24.6, 24.7 [2×CH(CH₃)₂], 33.2 (CH₂CH₂O), 43.0, 43.2 [2×CH(CH₃)₂], 55.2 (2×OCH₃), 57.7 (OCH₂CH₂CN), 64.3 (CH₂CH₂O), 66.5 (CHOP[NPr₂]₂), 71.0 (CH₂ODMT), 86.0 (OCPh₃), 104.0, 113.0, 121.0, 122.2, 125.4-130.1, 132.2, 136.1, 136.2, 144.9, 146.5, 155.3, 158.4 (aryl). ³¹P NMR (CDCl₃): δ 149.99, 150.09 in a 4:3 ratio. HRMS (ESI) m/z Calcd for C₄₇H₅₃N₄O₆PNa⁻ (MNa⁺) 823.3585 Found 823.3581.

### Example 10

### Oligonucleotide Synthesis, Purification, and Melting Temperature Determination

DMT-on oligodeoxynucleotides were carried out at 0.2 μmol scales on 500 Å CPG supports with an Expedite™ Nucleic Acid Synthesis System Model 8909 from Applied Biosystems with 1H-tetrazole as an activator for coupling reaction. The appropriate amidite (8a,b and 13) was dissolved in dry CH₂Cl₂ and inserted into the growing oligonucleotides chain using an extended coupling time (10 min). DMT-on oligonucleotides bound to CPG supports were treated with aqueous ammonia (32%, 1 ml) at room temperature and then at 55° C. over night. Purification of 5″-O-DMT-on ONs was accomplished by reversed-phase semipreparative HPLC on a Waters Xterra™ MS C₁₈ column with a Waters Delta Prep 4000 Preparative Chromatography System (Buffer A [0.05M triethylammonium acetate in H₂O (pH 7.4)] and Buffer B (75% MeCN in H₂O)). Flow 2.5 mL min⁻¹. Gradients: 2 min 100% A, linear gradient to 70% B in 38 min, linear gradient to 100% B in 3 min and then 100% A in 10 min) ODNs were DMT deprotected in 100 μL 80% acetic acid over 20 min. Afterwards, aqueous AcONa (1M, 50 μL) was added and the ONs were precipitated from EtOH (96%). All modified ODNs were confirmed by MALDI-TOF analysis on a Voyager Elite Bio spectroscopy Research Station from PerSeptive Biosystems. ODN Found m/z (Calculated m/z): ON2 4589.3 (4589.2), ON3 4580.1 (4581.3), ON4 4627.3 (4631.3), ON5 4476.5 (4481.1), ON7 4579.1 (4581.3), ON8 4629.2 (4631.3), ON9 4479.5 (4481.1), ON10 4591.7 (4581.3), ON11 4627.6 (4631.3), ON13 5042.7 (5040.7), ON14 5138.2 (5140.8), ON16 4578.9 (4581.3), ON17 4576.8 (4581.3). The purity of the final TFOs was found to be over 90%, checked by ion-exchange chromatography using LaChrom system from Merck Hitachi on Genpak-Fax column (Waters). Melting temperature measurments were performed on a Perkin-Elmer UV/VIS spectrometer Lambda 35 fitted with a PTP-6 temperature programmer. The triplexes were formed by first mixing the two strands of the Watson-Crick duplex, each at a concentration of 1.0 μM, followed by addition of the third (TFO) strand at a concentration of 1.5 μM in a buffer consisting of sodium cacodylate (20 mM), NaCl (100 mM), and MgCl₂ (10 mM) at pH 6.0 or 7.2. Parallel and antiparallel duplexes were formed by mixing of complementary ONs, each at a concentration of 1.0 μM, in the cacodylate buffer described above. Antiparallel duplex were formed by mixing of complementary ONs, each at a concentration of 1.0 μM in sodium phosphate buffer (10 mM) containing NaCl (140 mM) and EDTA (1 mM) at pH 7.0. The solutions were heated to 80° C. for 5 min and cooled to 5° C. and were then kept at this temperature for 30 min The melting temperature (Tₘ, ° C.) was determined as the maximum of the first derivative plots of the melting curves obtained by absorbance at 260 nm against increasing temperature (1.0° C./min). If needed experiments were also done at 373 nm. All melting temperatures are within the uncertainly ±1.0° C. as determined by repetitive experiments.

### Example 11

Fluorescence measurements. The fluorescence measurments were measured on a Perkin-Elmer LS-55 luminescence spectrometer fitted with a julabo F25 temperature controller set at 10° C. in the buffer 20 mM sodium cacodylate, 100 mM NaCl, and 10 mM MgCl₂ at pH 6.0. The triplexes and duplexes were formed in the same way as for Tₘ measurements except that only 1.0 μM of TFOs were used in all cases. The excitation wave length was set to 373 nm. Excitation and emission slits were set to 4 nm and 0.0 nm, respectively. The 0.0 nm slit is not completely closed and allowed sufficient light to pass for the measurement.

### Example 12

Molecular Modeling. Molecular modeling was performed with Macro Model v9.1 from Schrödinger. All calculations were conducted with AMBER* force field and the GB/SA water model. The dynamic simulations were preformed with stochastic dynamics, a SHAKE algorithm to constrain bonds to hydrogen, time step of 1.5 fs and simulation temperature of 300 K. Simulation for 0.5 ns with an equilibration time of 150 ps generated 250 structures, which all were minimized using the PRCG method with convergence threshold of 0.05 KJ/mol. The minimized structures were examined with Xcluster from Schrödinger, and representative low-energy structures were selected. The starting structures were generated with Insight II v97.2 from MSI, followed by incorporation of the modified nucleotide.

### Preparation of aryl imidazonaphthalimide analogues

### Example 13

### 3-Bromo-4-nitro-naphthalene-1,8-dicarboxylic anhydride (15)

Sodium nitrate (2.0 g, 23.5 mmol) was added to a solution of 4-bromo-naphthalene-1,8-dicarboxylic anhydride 1 (5.0 g, 18.1 mmol) in 98% H₂SO₄ (15 ml). The mixture was allowed to stand at 0-5° C. for 2.5 h, and the solution was poured into water and ice. The precipitate formed was filtered, washed with water, and dried. Recrystallization from AcOH gave 2 (5.0 g, 86%) as a long golden needles, mp 231-232° C. (231-232° C.)^{[42]}; ¹H NMR (DMSO-d₆): δ 8.18 (t, 1H, aryl), 8.73 (d, 1H, J=7.2 Hz, aryl), 8.82 (d, J=8.7, 1H, aryl), 8.90 (s, 1H, aryl). ¹³C NMR (DMSO-d₆): δ 120.3, 121.0, 121.7, 124.9, 125.4, 128.4, 130.9, 132.8, 134.8, 135.3, 158.9 (aryl). EI-MS: m/z 321 (100%, M⁺), 323 (97%).

### Example 14

### 3-Azido-4-nitro-naphthalene-1,8-dicarboxylic anhydride (16)

To a suspension of 2 (4.0 g, 12.48 mmol) in DMF (12 ml) was added a suspension of sodium azide (0.89 g, 13.72 mmol) in water (0.2 ml). The mixture was heated to 100° C. for 10 min and then poured into water and ice. The precipitate formed was filtered, washed with water, dried, and purified by silicagel column chromatography (ethyl acetate:petroleum ether 4:1) to afford compound 3 (3.0 g, 85%) was obtained as a yellow solid, mp 216-217° C.; ¹H NMR (DMSO-d₆): δ 8.04 (t, 1H, aryl), 8.69 (d, 1H, J=8.1 Hz, aryl), 8.85 (s, 1H, aryl), 8.88 (d, 1H, J=7.5, aryl). ¹³C NMR (DMSO-d₆): δ 115.7, 118.2, 119.8, 124.3, 125.0, 127.4, 129.3, 131.6, 135.2, 144.9, 159.1, 159.9 (aryl). IR (KBr, cm⁻¹) 2141.7, 1778.9, 1741.9; EI-MS: m/z 284 (100%, M⁺).

### Example 15

### 3,4-Diamino-naphthalene-1,8-dicarboxylic anhydride (17)

A mixture of 3 (1.25 g, 4.40 mmol) and 10% Pd/C (54 mg) in DMF (15 ml) was shaken in a Parr hydrogenator under hydrogen at 50 PSI pressure for 24 h. The catalyst was then filtered off and washed with DMF. The filtrate was concentrated, and water was added. The precipitate was then filtered, washed with water, and dried. Compound 4 (0.9 g, 91%) was obtained as a brown solid, mp>300° C.; ¹H NMR (DMSO-d₆): δ 5.30 (br s, 2H, NH₂), 6.88 (s, 2H, NH₂), 7.59 (t, 1H, aryl), 7.93 (s, 1H, aryl), 8.21 (d, 1H, J=7.2, aryl), 8.58 (d, 1H, J=8.7, aryl). ¹³C NMR (DMSO-d₆): δ 110.3, 118.0, 119.2, 121.4, 124.0, 126.2, 129.2, 129.3, 130.9, 131.6, 160.5, 162.1 (aryl). IR (KBr, cm⁻¹) 3372.9, 1736.4, 1622.9; EI-MS: m/z 228 (100%, M⁺).

### Example 16

### (S)-2,2-Dimethyl-4-(2-phenoxy ethyl)-9-phenyl-5,8-dihydrobenz[de]imidazo[4,5-g]isoquinoline-4,6-dione (18)

A mixture of diamine 4 (0.23 g, 1.0 mmol), (S)-4-(2-(2,2-dimethyl-1,3-dioxolan-4-yl)ethoxy)Benzaldehyde 5 and NaHSO₃ in DMF was heated at 100° C. until the reaction was completed (TLC). After the solution was cooled, water was added and then the precipitate was filtered. Recrystallization from DMF gave the corresponding anhydride 5 (0.44 g, 83%) as a brown solid, mp 230-233° C.; ¹H NMR (DMSO-d₆) δ 1.27 (s, 3H, CH₃), 1.33 (s, 3H, CH₃), 1.99 (m, 2H, CH₂CH₂O), 3.62 (m, 1H, CHH), 4.08-4.29 (m, 4H, CH, CHH, CH₂CH₂O), 7.12 (d, 2H, J=9.0 Hz, aryl), 7.87 (t, 1H, aryl), 8.11 (d, 2H, J=8.7 Hz, aryl), 8.37 (d, 1H, J=7.5 Hz, aryl), 8.48 (s, 1H, aryl), 8.82 (d, 1H, J=7.8 Hz, aryl). ¹³C NMR (DMSO-d₆): δ 25.6 (CH₃), 26.8 (CH₃), 32.9 (CH₂CH₂O), 64.8 (CH₂CH₂O), 68.7 (CH₂OC(CH₃)₂), 72.7 (CH₂CHCH₂), 107.9 (C(CH₃)₂), 111.7, 114.2, 114.8, 118.9, 121.2, 126.7, 126.8, 128.5, 128.6, 128.7, 129.0, 130.0, 131.3, 131.5, 132.0, 140.1, 154.2, 160.4, 160.8, 161.1 (aryl). HRMS (ESI) m/z Calcd for C₂₆H₂₃N₂O₆⁺ (MH⁺) 459.1550 Found 459.1553.

### Example 17

### (S)-2,2-Dimethyl-4-(2-phenoxy ethyl)-5-[2-(dimethylamino)propyl]-9-phenyl-5,8-dihydrobenz[de]imidazo[4,5-g]isoquinoline-4,6-dione (19)

A suspension of the corresponding anhydride 6 (0.40 g, 0.87 mmol) was treated with an excess of the 3-Dimethylamino-1-propylamin (0.22 g, 2.12 mmol) in absolute EtOH (25 ml). The mixture was heated at reflux temperature until the reaction was completed (TLC). After removal of organic solvent under reduced pressure, Compound 7 was obtained as solid which was used in the next step without further purification. (0.40 g, 84.5%) as a brown solid, mp 223-225° C.; ¹H NMR (DMSO-d₆) δ 1.27 (s, 3H, CH₃), 1.33 (s, 3H, CH₃), 1.77 (m, 2H, CH₂CH₂N(CH₃)₂), 2.01 (m, 2H, CH₂CH₂O), 2.24 (s, 6H, N(CH₃)₂), 2.32 (t, 2H, CH₂N(CH₃)₂), 3.61 (m, 1H, CHH), 4.04-4.15 (m, 6H, CH, CHH, CH₂CH₂O, CH₂CH₂CH₂N(CH₃)₂), 7.10 (d, 2H, J=8.4 Hz, aryl), 7.82 (t, 1H, aryl), 8.20 (d, 2H, J=8.7 Hz, aryl), 8.37 (d, 1H, J=6.9 Hz, aryl), 8.57 (s, 1H, aryl), 8.80 (d, 1H, J=7.8 Hz, aryl). ¹³C NMR (DMSO-d₆): δ 25.6 (CH₃), 25.8 [CH₂CH₂N(CH₃)₂] 26.8 (CH₃), 32.9 (CH₂CH₂O), 45.0 [N(CH₃)₂], 56.4 [CH₂CH₂CH₂N(CH₃)₂], 56.7 [CH₂N(CH₃)₂], 64.6 (CH₂CH₂O), 68.7 (CH₂OC(CH₃)₂), 72.7 (CH₂CHCH₂), 107.9 (C(CH₃)₂), 114.7, 115.0, 120.2, 122.2, 122.4, 122.8, 124.3, 126.0, 127.8, 128.3, 128.6, 128.7, 131.3, 131.5, 135.8, 141.8, 154.7, 159.9, 163.6, 163.7 (aryl). HRMS (ESI) m/z Calcd for C₃₁H₃₅N₄O₅⁺ (MH⁺) 543.2602 Found 543.2607.

### Example 18

### (S)-4-({4[(-5,8-Dihydrobenz[de]imidazol-2-yl)phenoxy}butane-1,2-diol-N-(dimethylamino)propyl][4,5-g]isoquinoline-4,6-dione (20)

Compound 7 (0.35 g, 0.65 mmol) was stirred in 80% acetic acid (20 ml) for 24 h at room temperature. The solvent was removed in vacuo, and the residue was coevaporated twice with toluene/EtOH (30 ml, 5:1, v/v). The residue was dried in vacuo to afford compound 8. Yield 0.32 g (100%) as brown solid, mp 69-70° C. which was used in the next step without further purification. ¹H NMR (DMSO-d₆): δ 1.77 (m, 3H, CH₂CH₂N(CH₃)₂, CHHCH₂O), 1.99 (m, 1H, CHHCH₂O), 2.20 (s, 6H, N(CH₃)₂), 2.30 (s, 2H, 2×OH), 2.37 (t, 2H, CH₂N(CH₃)₂) 3.41 (m, 2H, CHHOH and CHOH), 3.70 (m, 1H, CHHOH), 4.06 (m, 2H, CH₂CH₂O), 4.20 (m, 2H, CH₂CH₂CH₂N(CH₃)₂), 7.24 (d, 2H, J=6.9 Hz, aryl), 7.86 (t, 1H, aryl), 8.20 (d, 2H, J=7.5 Hz, aryl), 8.39 (d, 1H, J=7.5 Hz, aryl), 8.58 (s, 1H, aryl), 8.83 (d, 1H, J=7.8 Hz, aryl). ¹³C NMR (DMSO-d₆): δ 25.6 [CH₂CH₂N(CH₃)₂] 33.0 (CH₂CH₂O), 44.7 [N(CH₃)₂], 56.1 [CH₂CH₂CH₂N(CH₃)₂], 56.5 [CH₂N(CH₃)₂], 64.9 (CH₂CH₂O), 66.0 (CH₂OH), 68.0 (CHOH), 114.9, 115.8, 119.7, 121.6, 122.3, 124.4, 125.2, 126.4, 127.8, 128.1, 128.4, 128.6, 128.8, 131.4, 131.5, 153.6, 160.5, 163.5, 163.7 (aryl). HRMS (ESI) m/z Calcd for C₂₈H₃₁N₄O₅⁺ (MH⁺) 503.2289 Found 503.2297.

### Example 19

### (S)-5-[2-(dimethylamino)propyl]-9-phenyl-5,8-dihydrobenz[de]imidazo[4,5-g]isoquinoline-4,6-dione-1-(bis(4-methoxyphenyl)(phenyl)methoxy)butan-2-ol (21)

Compound 8 (0.25 g, 0.50 mmol) was dissolved in dry pyridine (20 ml) and 4,4′-dimethoxytrityl chloride (DMT-Cl) (0.20 g, 0.60 mmol) was added under a nitrogen atmosphere. The reaction mixture was stirred for 24 h at room temperature. The solvent was evaporated off under reduced pressure, and the residue was purified by silica gel column chromatography [EtOAc/NEt₃ (100:2, v/v)] affording compound 9. Yield 0.30 g (75%) as yellow foam. ¹H NMR (CDCl₃): δ 1.78 (m, 3H, CH₂CH₂N(CH₃)₂, CHHCH₂O), 2.20 (s, 6H, N(CH₃)₂), 1.99 (m, 1H, CHHCH₂O), 2.33 (s, 1H, OH), 2.58 (t, 2H, CH₂N(CH₃)₂), 2.94 (m, 2H, CH₂ODMT), 3.27 (m, 1H, CHOH) 3.78 (s, 6H, 2×OCH₃), 4.15 (m, 2H, CH₂CH₂O), 4.24 (m, 2H, CH₂CH₂CH₂N(CH₃)₂), 6.83 (d, 4H, J=8.1 Hz, DMT), 6.95 (d, 2H, J=8.7 Hz, aryl), 7.30-7.35 (m, 7H, aryl), 7.45 (d, 2H, J=6.3 Hz, aryl), 7.76 (t, 1H, aryl), 8.35 (d, 2H, J=7.5 Hz, aryl), 8.52 (d, 1H, J=7.5 Hz, aryl), 8.87 (s, 1H, aryl), 9.06 (d, 1H, J=8.7 Hz, aryl). ¹³C NMR (CDCl₃): δ 26.2 [CH₂CH₂N(CH₃)₂], 33.0 (CH₂CH₂O), 45.4 [N(CH₃)₂], 55.1 (2×OCH₃), 57.3 [CH₂CH₂CH₂N(CH₃)₂], 58.2 (CH₂N(CH₃)₂], 69.8 (CH₂CH₂O), 70.5 (CHOH), 71.0 (CH₂ODMT), 86.2 (OCPh₃), 112.9, 113.2, 125.9-130.3, 131.8, 132.1, 138.7, 143.4, 145.3, 146.8, 157.6, 158.3, 158.4 (aryl). HRMS (ESI) m/z Calcd for C₄₉H₄₉N₄O₇⁺ (MH⁺) 805.3595 Found 805.3580.

### Example 20

### (S)-5-[2-(dimethylamino)propyl]-9-phenyl-5,8-dihydrobenz[de]imidazo[4,5-g]isoquinoline-4,6-dione-1-(bis(4-methoxyphenyl)(phenyl)-methoxy)butan-2-yl 2-cyanoethyl diisopropyl-phosphoramidite (22)

Compound 9 (0.20 g, 0.25 mmol) was dissolved under an argon atmosphere in anhydrous CH₂Cl₂ (15 ml). N,N′-Diisopropyl ammonium tetrazolide (0.065 g, 0.38 mmol) was added, followed by dropwise addition of 2-cyanoethyl tetraisopropylphosphordiamidite (0.23 g, 0.75 mmol) under external cooling with an ice-water bath. The reaction mixture was stirred at room temperature under an argon atmosphere overnight. After 24 h, analytical TLC showed no more starting material. The solvent was evaporated under reduced pressure and the residue was purified by silica gel column chromatography [EtOAc/NEt₃ (100:2, v/v)] affording compound 10. Yield: 0.20 g (80%) as yellow oil. HRMS (ESI) m/z Calcd for C₅₈H₆₆N₆O₈P⁺ (MH⁺) 1005.4675 Found 1005.4630.

### REFERENCES

[1] Vasquez, K. M.; Wilson, J. M. Trends Biochem. Sci. 1998, 23, 4.

[2] Maher, L. J. Cancer Invest. 1996, 14, 66.

[3] Neidle, S. Anti-Cancer Drug Des. 1997, 12, 433.

[4] Wang, G.; Levy, D. D.; Seidman, M. M.; Glazer, P. M. Mol. Cell. Biol. 1995, 15, 1759.

[5] Majumdar, A.; Khorlin, A.; Dyatkina, N. B.; Lin, M. F.; Powell, J.; Fei, Z.; Khripine, Y.; Watanabe, K. A.; George, J.; Glazer, P. M.; Seidman, M. M. Nat. Genet. 1998, 20, 212.

[6] Vasquez, K. M.; Naryanan, L.; Glazer, P. M. Science 2000, 290, 530.

[7] Guntaka, R. V.; Varma, B. R.; Weber, K. T. Int. J. Biochem. Cell Biol. 2003, 35, 22.

[8] Casey, B. P.; Glazer, P. M. Nucleic Acid Res. Mol. Biol. 2001, 67, 163.

[9] Knauert, M. P.; Glazer, P. M. Hum. Mol. Genet. 2001, 10, 2243.

[10] Seidman, M. M.; Glazer, P. M. J. Clin. Invest. 2003, 112, 487.

[11] Roig, V.; Asseline, U. J. Am. Chem. Soc. 2003, 125, 4416.

[12] Hildbrand, S.; Blaser, A.; Parel, S. P.; Leumann, C. J. J. Am. Chem. Soc. 1997, 119, 5499.

[13] Xodo, L. E.; Manzini, G.; Quadrifoglio, F.; van der Marcel, G. A.; Van Boom, J. H. Nucleic Acids Res. 1991, 19, 5625.

[14] Carlomagno, T.; Blommers, M. J. J.; Meiler, J.; Cuenoud, B.; Griesinger, C. J. Am. Chem. Soc. 2001, 123, 7364.

[15] Cuenoud, B.; Casset, F.; Hüsken, D.; Natt, F.; Wolf, R. M.; Altmann, K.-H.; Martin, P.; Moser, H. E. Angew. Chem. Int. Ed. 1998, 37, 1288.

[16] Wengel, J. Acc. Chem. Res. 1999, 32, 301.

[17] Obika, S.; Hari, Y.; Sugimoto, T.; Sekiguchi, M.; Imanishi, T. Tetrahedron Let. 2000, 41, 8923.

[18] Sun, B.-W.; Babu, B. R.; Sorensen, M. D.; Zakrzewska, K.; Wengel, J.; Sun, J.-S. Biochemistry, 2004, 43, 4160.

[19] Basye, J.; Trent, J. O.; Gao, D.; Ebbinghaus, S. W. Nucleic Acids Res. 2001, 29, 4873.

[20] Michel, T.; Debart, F.; Heitz, F.; Vasseur, J.-J. Chem Bio Chem. 2005, 6, 1254.

[21] Ehrenmann, F.; Vasseur, J.-J.; Debart, F. Nucl. Nucl. Nucleic Acids. 2001, 20, 797.

[22] Michel, T.; Debart, F.; Vasseur, J.; Geinguenaud, F.; Taillandier, E. J. Biomol. Struct. Dyn. 2003, 21, 435.

[23] Tereshko, V.; Gryaznov, S. M.; Egli, M. J. Am. Chem. Soc. 1998, 120, 269.

[24] Escudé, C.; Giovannangeli, C.; Sun, J. S.; Lloyd, D. H.; Chen, J.-K.; Gryaznov, S. M.; Garestier, T.; Hélène, C. Proc. Natl. Acad. Sci. U.S.A. 1996, 93, 4365.

[25] Gryaznov, S. M.; Lloyd, D. H.; Chen, J.-K.; Schultz, R. G.; DeDionisio, L. A.; Ratmeyer, L.; Wilson, W. D. Proc. Natl. Acad. Sci. U.S.A. 1995, 92, 5798.

[26] Gryaznov, S. M.; Chen, J.-K. J. Am. Chem. Soc. 1994, 116, 3143.

[27] Chur, A.; Holst, B.; Dahl, O.; Valentin-Hansen, P.; Pedersen, E. B. Nucleic Acids Res. 1993, 21, 5179.

[28] a) Filichev, V. V.; Pedersen, E. B. J. Am. Chem. Soc. 2005, 127, 14849. b) Filichev, V.

V.; Gaber, H.; Olsen, T. R.; Jorgensen, P. T.; Jessen, C. H.; Pedersen, E. B. Eur. J. Org. Chem. 2006, 3960. c) Globisch, D.; Bomholt, N.; Filichev, V. V.; Pedersen, E. B. Helv. Chim. Acta, 2008, 91, 805.

[29] Paramasivam, M.; Cogoi, S.; Filichev, V. V.; Bomholt, N.; Pedersen, E. B.; Xodo, L. E. Nucleic Acids Res. 2008, 36, 3494.

[30] Elovaara, E.; Mikkola, J.; Stockmann-Juvala, H.; Luukkanen, L.; Keski-Hynnilä, H.; Kostianinen, R.; Pasanen, M.; Pelkonen, O.; Vainio, H. Arch. Toxicol. 2007, 81, 169.

[31] Chau, A.; Cote, B.; Ducharme, Y.; Frenette, R.; Friesen, R.; Gagnon, M.; Giroux, A.; Martins, E.; Yu, H.; Wu, T. 2007 WO 2007/095753.

[32] Kim, B. Y.; Ahn, J. B.; Lee, H. W.; Moon, K. S.; Sim, T. B.; Shin, J. S.; Ahn, S. K.; Hong, C. I. Chem. Pharm. Bull. 2003, 51, 276.

[33] Krebs, F. C.; Spanggaard, H. J. Org. Chem. 2002, 67, 7185.

[34] Nakamura, T.; Sato, M.; Kakinuma, H.; Miyata, N.; Taniguchi, K.; Bando, K.; Koda, A.; Kameo, K. J. Med. Chem. 2003, 46, 5416.

[35] Felsenfeld, G.; Miles, H. T. Annu. Rev. Biochem. 1967, 36, 407.

[36] Liu, K.; Miles, H. T.; Frazier, J.; Sasisekharan, V. Biochemistry 1993, 32, 11802.

[37] Zhou, B.-W.; Puga, E.; Sun, J.-S.; Garestier, T.; Hélène, C. J. Am. Chem. Soc. 1995, 117, 10425.

[38] Ren, R. X.-F.; Chaudhuri, N. C.; Paris, P. L.; Rumney, I. S.; Kool, E. T. J. Am. Chem. Soc. 1996, 118, 7671.

[39] Guckian, K. M.; Schweitzer, B. A.; Ren, R. X.-F.; Sheils, C. J.; Paris, P. L.; Tahmassebi, D. C.; Kool, E. T. J. Am. Chem. Soc. 1996, 118, 8182.

[40] WO06125447A2

[41] TIMOFEEV et al. Methidium Intercalator Inserted into Synthetic Oligonucleotides. Tetrahedron Letters, 1996, vol. 37, No. 47, 8467-8470.

[42] Kadhim, A. D.; Peters, A. T. J. Soc. Dyers Colourists. 1974, 90, 153.

1. An intercalating oligonucleotide for stabilizing natural or modified DNA and RNA triplexes, duplexes and hybrids thereof having the general structure (I): 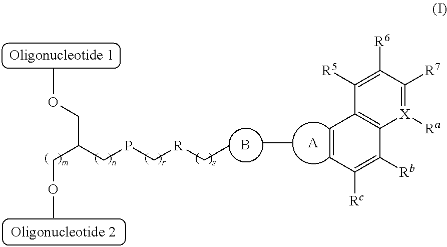
wherein
R^{a} and R^{b} together form 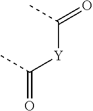
R^{c} is H
or
R^{b} and R^{c} together form 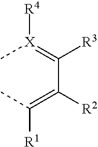
R^{a}═R⁸
A is a 5-, 6-, or 7-membered heteroaromatic ring, containing at least one heteroatom selected from nitrogen, oxygen and sulfur, especially one nitrogen atom and at least one further heteroatom selected from nitrogen, substituted nitrogen, oxygen and sulfur,
wherein B is a monocyclic or polycyclic aromatic ring systems optionally selected from the group of 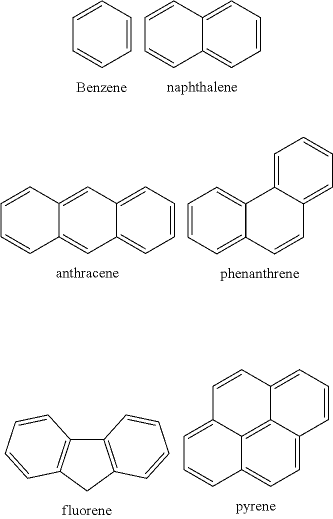
and monocyclic or bicyclic heteromatic ring systems optionally selected from the group of 5-membered aromatic heterocyclic rings and 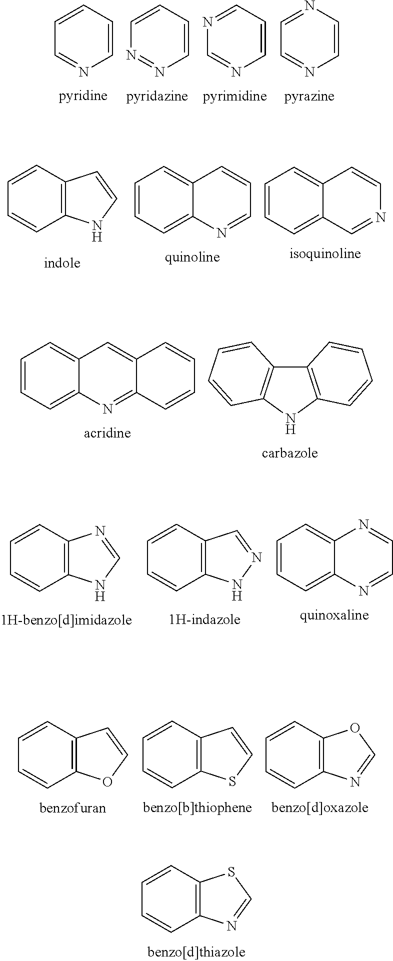
wherein
P and R are independently of each other selected from the group consisting of O, S, NR⁹, —CH₂, —CH—, —C≡C—, wherein R⁹ is hydrogen, methyl, ethyl, or hydroxyl,
m is 0 or 1, n, r, s are independently of each other 0, 1, 2 or 3, especially 0, 1 or 2,
Oligonucleotide 1 and Oligonucleotide 2 are defined independently of each other oligonucleotide consisting of subunits of DNA, RNA, PNA, HNA, MNA, ANA, FANA, LNA, CAN, INA, CeNA, TNA, (2′-NH)-TNA, (3′-NH)-TNA, α-L-Ribo-LNA, α-L-Xylo-LNA, β-D-Ribo-LNA, β-D-Xylo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-RRNA, 2′-OR-RNA, 2′-AE-RNA, α-L-RNA, β-D-RNA, and modifications thereof,
R¹, R², R³, R⁴ R⁵, R⁶, R⁷ and R⁸ are independently of each other hydrogen, halogen, C₁-C₁₈alkyl, C₁-C₁₈alkyl which is substituted by E and/or interrupted by D, C₂-C₁₈alkenyl, C₂-C₁₈alkynyl, C₁-C₁₈alkoxy, C₁-C₁₈alkoxy which is substituted by E and/or interrupted by D, C₆-C₂₄aryl, C₆-C₂₄aryl which is substituted by G, C₂-C₂₀heteroaryl, C₂-C₂₀heteroaryl which is substituted by G, C₇-C₂₅arakyl,
or two substituents R¹ and R², R² and R³, R³ and R⁴, R⁵ and R⁶, R⁶ and R⁷, R⁷ and R⁸ which are adjacent to each other, together form a group 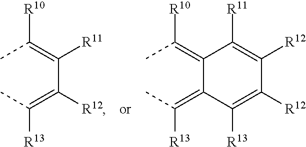
or two substituents R⁴ and R⁸, which are adjacent to each other, together form a group 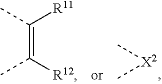
wherein R¹⁰, R¹¹, R¹², R¹³ are independently of each other hydrogen, halogen, C₁-C₁₈alkyl, C₁-C₁₈alkyl which is substituted by E and/or interrupted by D, C₂-C₁₈alkenyl; C₂-C₁₈alkynyl, C₁-C₁₈alkoxy, C₁-C₁₈alkoxy which is substituted by E and/or interrupted by D, C₆-C₂₄aryl, C₆-C₂₄aryl which is substituted by G, C₂-C₂₀heteroaryl, C₂-C₂₀heteroaryl which is substituted by G, C₇-C₂₅aralkyl;
X² is O, S, C(R¹⁴)(R¹⁵), or N—R¹⁶, wherein R¹⁶ is hydrogen, hydroxyl, C₁-C₁₈alkyl, C₁-C₁₈alkyl which is substituted by E and/or interrupted by D, C₂-C₁₈alkenyl, C₂-C₁₈alkynyl which is substituted by E and/or interrupted by D, C₁-C₁₈alkoxy, C₁-C₁₈alkoxy which is substituted by E and/or interrupted by D, C₁-C₁₈aminoalkyl, C₁-C₁₈aminoalkyl which is substituted by E and/or interrupted by D, C₅-C₁₈cycloalkyl, C₅-C₁₈cycloalkyl which is substituted by E and/or interrupted by D, C₆-C₁₈aryl, C₂-C₂₀heteroaryl, C₆-C₁₈aryl, or C₂-C₂₀heteroaryl, which are substituted by C₁-C₁₈alkyl, or C₁-C₁₈alkoxy; C₁-C₁₈alkyl; or C₁-C₁₈alkyl which is interrupted by —O—,
R¹⁴ and R¹⁵ together form a group of formula ═CR¹⁷R¹⁸, wherein R¹⁷ and R¹⁸ are independently of each other hydrogen, C₁-C₁₈alkyl, C₁-C₁₈alkyl which is substituted by E and/or interrupted by D, C₆-C₂₄aryl, C₆-C₂₄aryl which is substituted by G, C₂-C₂₀heteroaryl, or C₂-C₂₀heteroaryl which is substituted by G, or R¹⁴ and R¹⁵ together form a five or six membered ring, which can be substituted by C₁-C₁₈alkyl, C₁-C₁₈alkyl which is substituted by E and/or interrupted by D, C₆-C₂₄aryl, C₆-C₂₄aryl which is substituted by G, C₂-C₂₀heteroaryl, or C₂-C₂₀heteroaryl which is substituted by G, C₂-C₁₈alkenyl; C₂-C₁₈alkynyl, C₁-C₁₈alkoxy, C₁-C₁₈alkoxy which is substituted by E and/or interrupted by D, C₇-C₂₅aralkyl, or —C(═O)—R¹⁹, wherein R¹⁹ is hydrogen, C₆-C₁₈aryl, C₆-C₁₈aryl which is substituted by C₁-C₁₈alkyl, or C₁-C₁₈alkoxy, C₁-C₁₈alkyl, or C₁-C₁₈alkyl which is interrupted by —O—,
D is —CO—, —S—, —SO—, —SO₂, —O—, —NR²⁰—, —SiR²¹R²²—, —POR²³—, —CR²⁴═CR²⁵—, or —C≡C—; and
E is —OR²⁶, —SR²⁶, —COR²⁶, —NR²⁰R²⁷, CN, or halogen,
G is E, C₁-C₁₈alkyl, C₁-C₁₈alkyl which is interrupted by D, C₁-C₁₈alkoxy, or C₁-C₁₈alkoxy which is substituted by E and/or interrupted by D, wherein
R²⁰, R²⁴, R²⁵, R²⁷ are independently of each other hydrogen, C₁-C₁₈alkyl, C₆-C₁₈aryl, C₆-C₁₈aryl which is substituted by C₁-C₁₈alkyl, or C₁-C₁₈alkoxy, C₁-C₁₈alkyl, or C₁-C₁₈alkyl which is interrupted by —O—, or 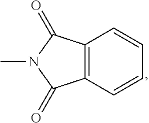
R²⁰ and R²⁷ together form a five or six membered ring, in particular 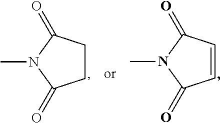
R²¹, R²² and R²³are independently of each other C₁-C₁₈alkyl, C₆-C₁₈aryl, or C₆-C₁₈aryl, which is substituted by C₁-C₁₈alkyl, and
R²⁶ is independently of each other hydrogen, C₁-C₁₈alkyl, C₆-C₁₈aryl, C₆-C₁₈aryl which is substituted by C₁-C₁₈alkyl, or C₁-C₁₈alkoxy, C₁-C₁₈alkyl, or C₁-C₁₈alkyl which is interrupted by —O—,
X is C or N with the proviso that when X is CH or N then the nitrogen atom is unsubstituted, and
Y is O or N—R²⁸, wherein R²⁸ is hydrogen, methyl, ethyl, hydroxyl, alkyl, substituted alkyl, alkoxy, substituted alkoxy, aminoalkyl, substituted aminoalkyl, cycloalkyl, substituted cycloalkyl, alkenyl, substituted alkenyl, alkynyl, substituted alkynyl, aryl, substituted aryl, heterocyclic, and substituted heterocyclic.

2. An intercalating oligonucleotide according to claim 1, having any one of the general structures (IIa-IId): 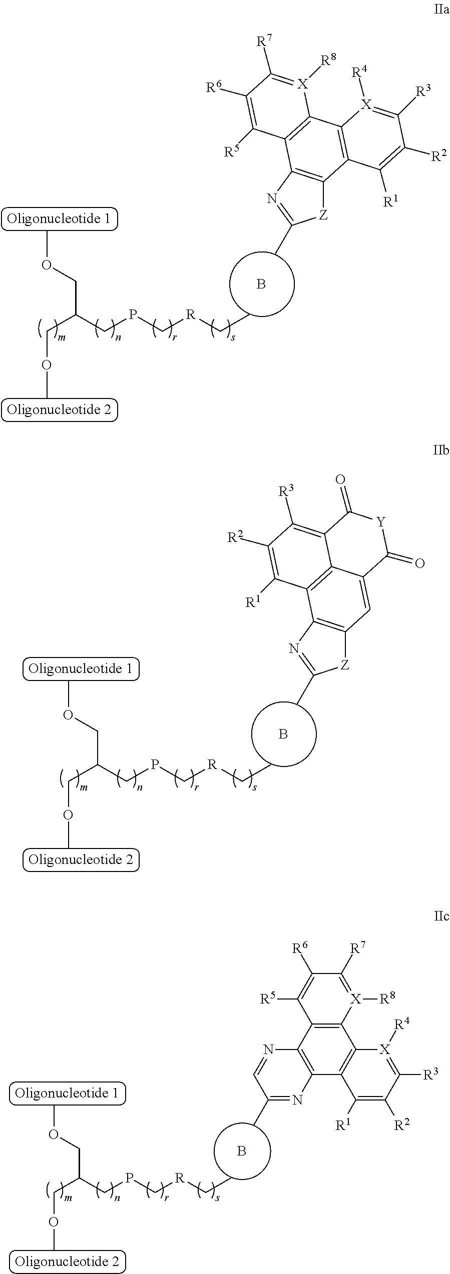 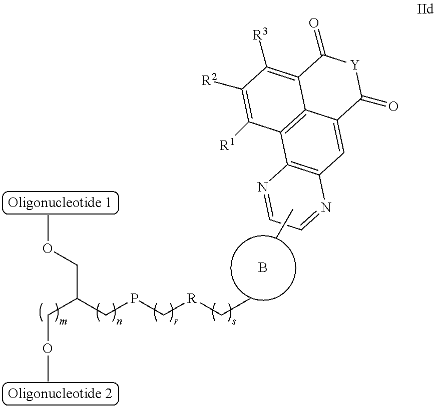
Z is O, S or N—R²⁸, wherein R²⁸ is as defined in claim 1

3. An intercalating oligonucleotide according to claim 1, wherein backbone monomer comprises 1-O-methyleneglycerol or 1,2-dihydroxybutoxy, said oligonucleotide selected from the general structures (IIIa-IIIh):

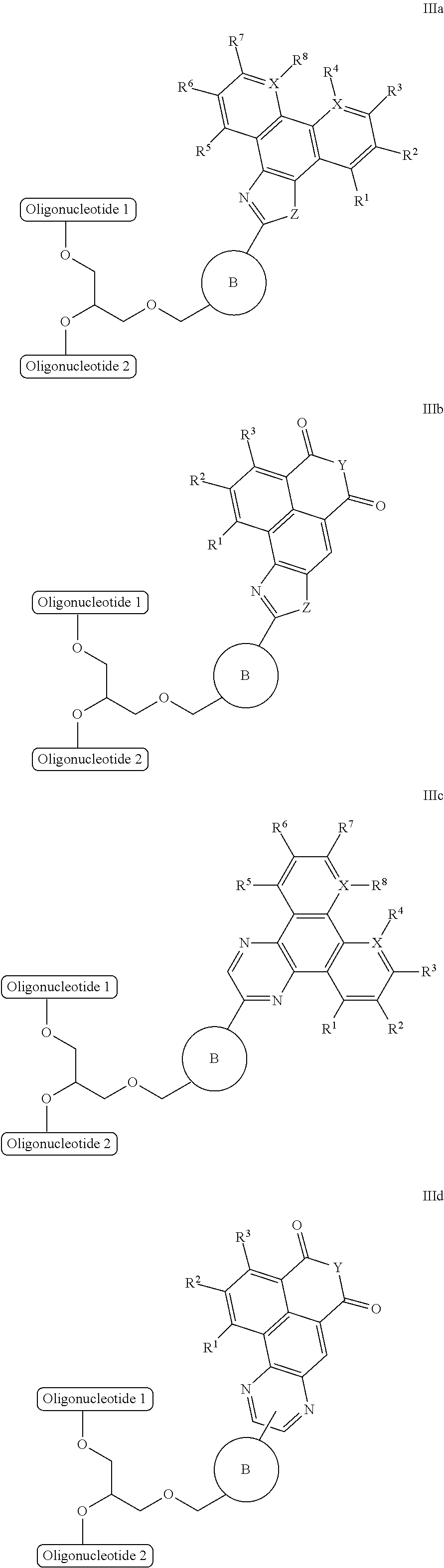

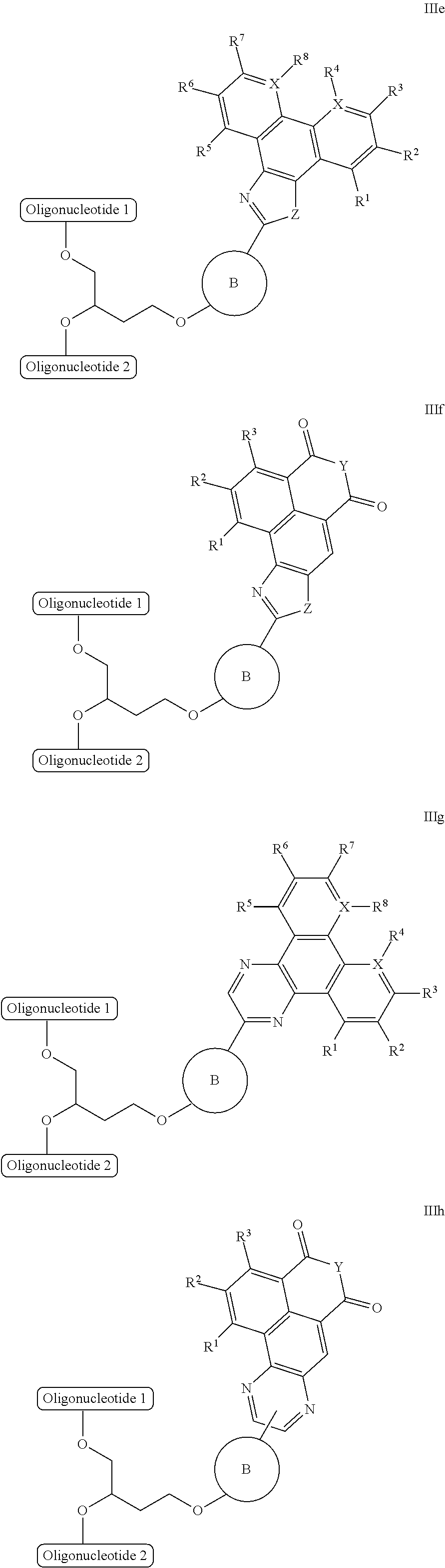

4. An intercalating oligonucleotide according to claim 3, wherein B consists of meta-, ortho- or para-substituted phenyl ring, said oligonucleotide selected from the general structures (IVa-IVh):

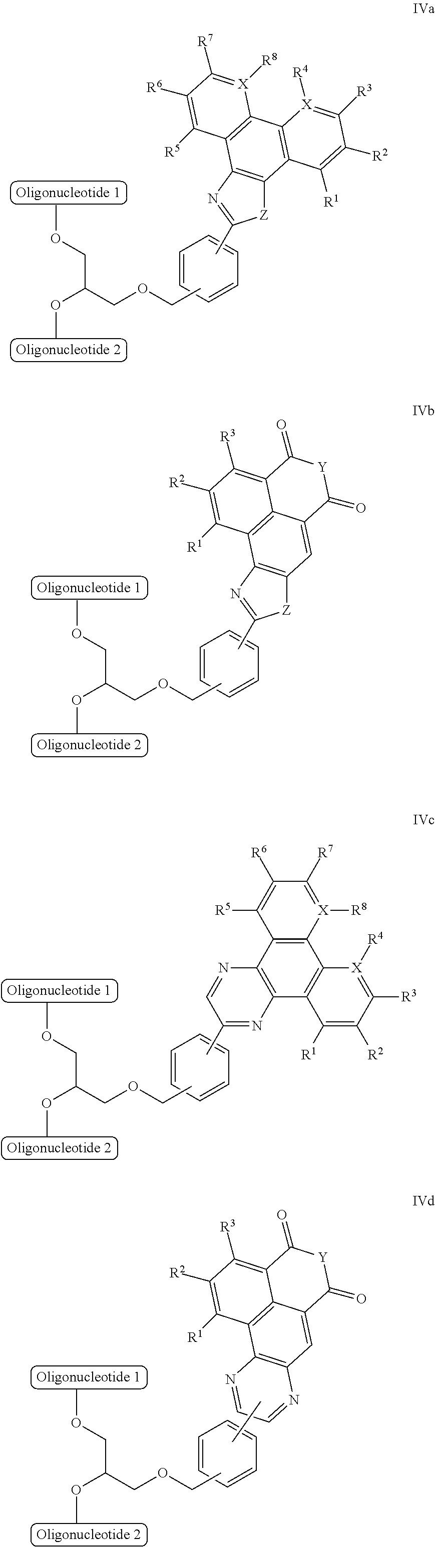

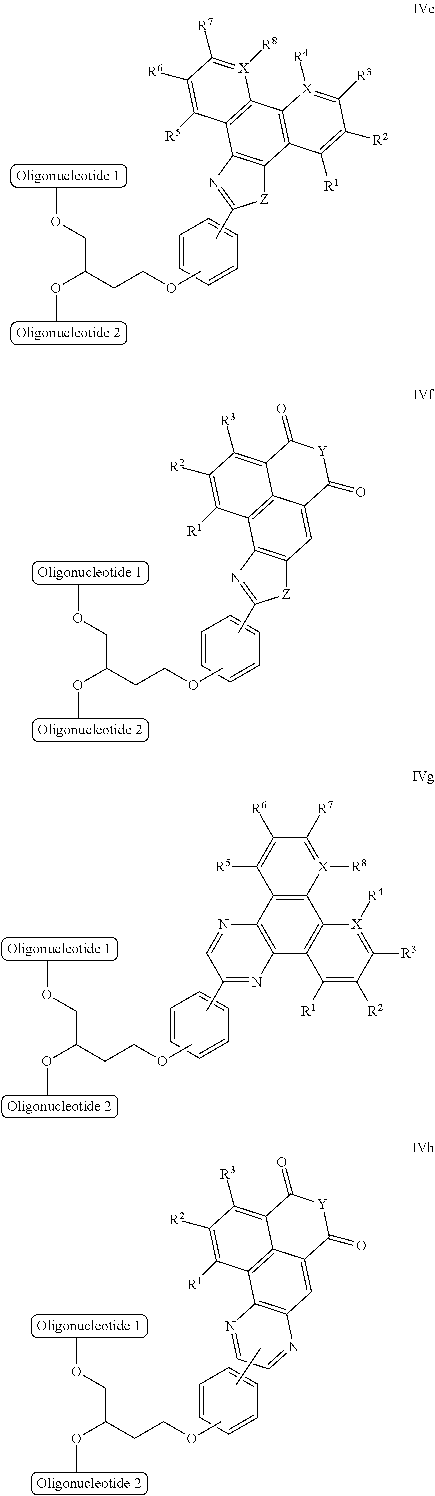

5. An intercalating oligonucleotide according to claim 1, wherein substituted ethyleneglycol is pure stereoisomer (R) or (S).

6. An intercalating oligonucleotide according to claim 1 having the structures (Va-Vh):

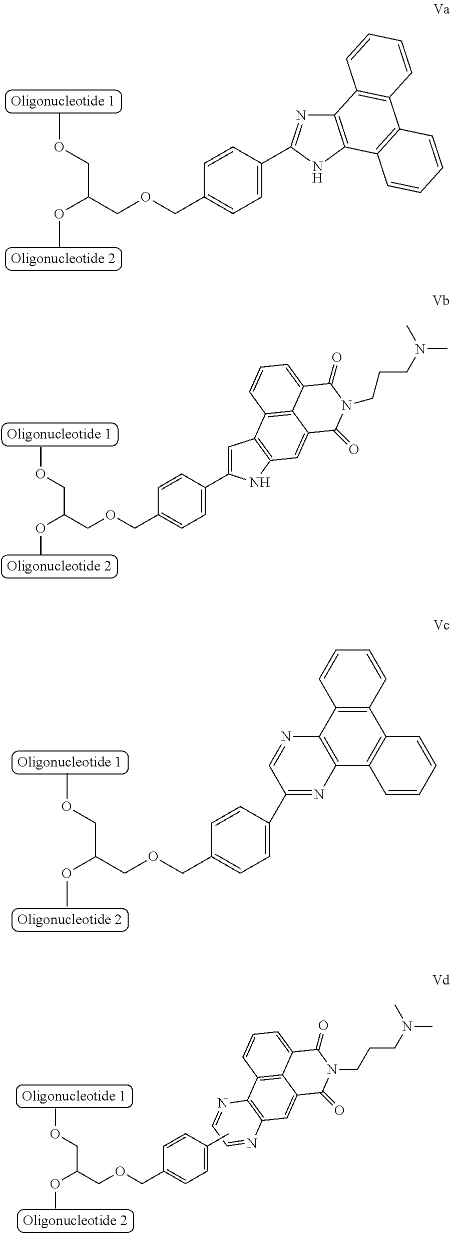

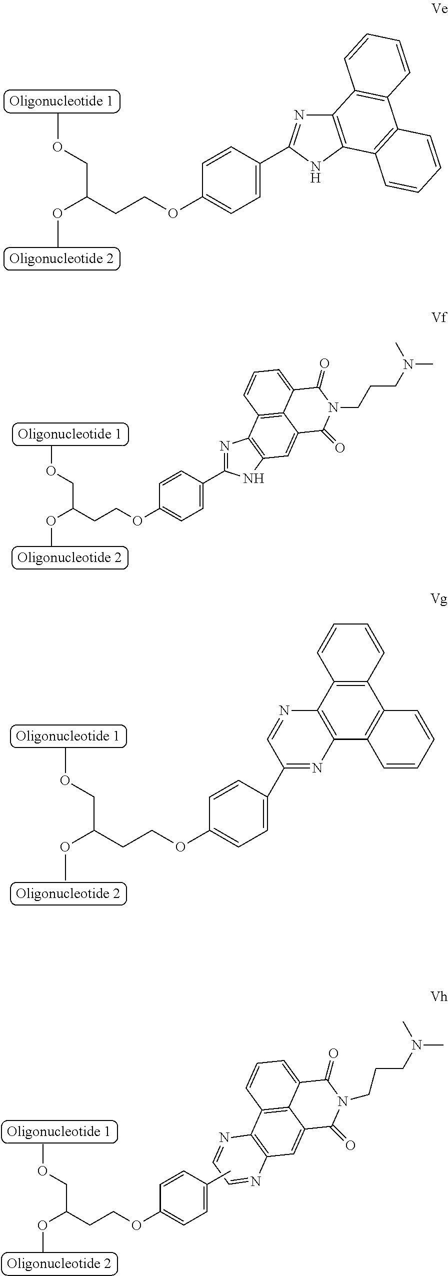

7. An intercalating oligonucleotide according to claim 1, wherein Oligonucleotide 1 and Oligonucleotide 2 independently of each other are single-stranded pyrimidin-rich oligonucleotides consisting of subunits of DNA, RNA, PNA, HNA, MNA, ANA, FANA, LNA, CAN, INA, CeNA, TNA, (2′-NH)-TNA, (3′-NH)-TNA, α-L-Ribo-LNA, α-L-Xylo-LNA, β-D-Ribo-LNA, β-D-Xylo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-RRNA, 2′-OR-RNA, 2′-AE-RNA, α-L-RNA, β-D-RNA, and modifications thereof or single-stranded pyrimidin-rich oligoribonucleotides consisting of subunits of DNA, RNA, PNA, HNA, MNA, ANA, FANA, LNA, CAN, INA, CeNA, TNA, (2′-NH)-TNA, (3′-NH)-TNA, α-L-Ribo-LNA, α-L-Xylo-LNA, β-D-Ribo-LNA, β-D-Xylo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-RRNA, 2′-OR-RNA, 2′-AE-RNA, α-L-RNA, β-D-RNA, and modifications thereof or single-stranded purine-rich oligonucleotides consisting of subunits of DNA, RNA, PNA, HNA, MNA, ANA, FANA, LNA, CAN, INA, CeNA, TNA, (2′-NH)-TNA, (3′-NH)-TNA, α-L-Ribo-LNA, α-L-Xylo-LNA, β-D-Ribo-LNA, β-D-Xylo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-RRNA, 2′-OR-RNA, 2′-AE-RNA, α-L-RNA, β-D-RNA, and modifications thereof or single-stranded purine-rich oligoribonucleotides.

8. A pharmaceutical composition suitable for use in antisense therapy and antigene therapy, said composition comprising an intercalating oligonucleotide of claim 1.
